(19) United States
(12) Patent Application Publication
(10) Pub. No.: US 2023/0309833 A1
(43) Pub. Date: Oct. 5, 2023

(54) METHODS AND MATERIALS FOR USING [18F]-F-ARAG IN CARDIAC IMAGING

(71) Applicant: CELLSIGHT TECHNOLOGIES INC., San Francisco, CA (US)

(72) Inventor: Jelena Levi, San Francisco, CA (US)

(73) Assignee: CELLSIGHT TECHNOLOGIES INC., San Francisco, CA (US)

(21) Appl. No.: 18/007,239

(22) PCT Filed: Jul. 28, 2021
(86) PCT No.: PCT/US2021/043431

§ 371 Date: Jan. 27, 2023

Related U.S. Application Data
(60) Provisional application No. 63/057,643, filed on Jul. 28, 2020.

(51) Int. Cl. A61B 5/00; A61B 8/08

(52) U.S. Cl. CPC A61B 5/0044; A61B 8/0883; A61B 2576/023

(57) ABSTRACT

Embodiments of the present disclosure provide compositions and methods for performing positron emission tomography (PET) and, more particularly, to compositions and methods for the development and use of ¹⁸F-based PET tracers for use in selected imaging techniques such as studies of myocardial physiology. In particular, as disclosed herein, at tracer levels, [¹⁸F]F—AraG has the ability to be used in a number of new PET methodologies including those designed for cardiac and/or mitochondrial activity imaging. The use of [¹⁸F]F—AraG as a PET tracer offers significant advantages over conventional ¹⁸F labeled tracers in a number of distinct applications including observations of selected physiological phenomena such as myocardial perfusion, myocardial viability, and heart inflammation. Methods of the invention include those designed to use [¹⁸F]F—AraG to observe patient physiological responses to various therapeutic agents, as well as using [¹⁸F]F—AraG as a PET tracer in drug development studies.

### CROSS-REFERENCE TO RELATED APPLICATIONS

This application claims the benefit under 35 U.S.C. Section 119(e) of co-pending and commonly-assigned U.S. Provisional Pat. Application Serial No 63/057,643, filed on Jul. 28, 2020, and entitled “METHODS AND MATERIALS FOR USING [18F]-F-AraG IN CARDIAC IMAGING” which application is incorporated by reference herein. This application is related to co-pending U.S. Application entitled “Compounds and Methods of Making Compounds”, having serial number 17/185,502, filed on Feb. 25, 2021, the contents of which are incorporated herein by reference.

### BACKGROUND

Cardiovascular disease (CVD) has been the leading killer of Americans for decades. In addition, CVD has become America’s costliest chronic disease. In 2018, stroke and heart failure were the most expensive chronic conditions in the Medicare fee-for-service program.

The instant disclosure relates, in general, to positron emission tomography (PET) and, more particularly, to compositions and methods for the development and use of ¹⁸F-based PET tracers for use in cardiac imaging techniques such as myocardial perfusion studies. The long physical half-life (109 minutes) of ¹⁸F-based tracers allows for clinical studies without the necessity of an on-site cyclotron. In addition, with contemporary PET camera technology, quantitative measurements of myocardial radioactivity concentration can be made with high temporal sampling and good statistical precision.

Cardiac imaging techniques play a central role in the noninvasive diagnosis and risk assessment of CVD, and associated decisions made by medical practitioners on how to optimally manage CVD in different individuals. The development of new ¹⁸F-labeled tracers useful in positron emission tomography methods of cardiac imaging can provide artisans with additional opportunities to manage CVD, and to further extend the range of clinical studies. In view of the enormous public health burden of cardiovascular disease, there is a need in the art for additional compositions and methods that are designed to use ¹⁸F-based PET tracers in cardiac imaging techniques.

### SUMMARY

The compound used in the methods disclosed herein, [18F]—F—arabinofuranosyl guanine was initially developed as a PET imaging agent for activated T cells. This compound is a ¹⁸F-labeled analog of arabinofuranosyl guanine (AraG), one that can be phosphorylated, and trapped intracellularly, by two kinases: cytoplasmic deoxycytidine kinase (dCK) and deoxyguanosine kinase (dGK). As discussed below, we have discovered that, at tracer levels, [¹⁸F]F—AraG has the ability to be used in a number of new PET methodologies including those designed for cardiac and/or mitochondrial activity imaging. Heart, with its high energy needs, is an organ particularly rich in mitochondria. In the past decade, mitochondrial dysfunction has been recognized as an important aspect of cardiovascular pathology. Consequently, treatments focusing on observing and improving heart’s mitochondrial activity are a focus of biomedical research.

The disclosure provided herein is based in part upon on the discovery that [¹⁸F]F—AraG is an agent that is well suited to observe certain physiological phenomena, including for example, mitochondrial activity in cells of the heart. In view of this, embodiments of the invention provide methods for using [¹⁸F]F—AraG in cardiac imaging. In illustrative embodiments of the invention, the use of [¹⁸F]F—AraG as a PET tracer has been evaluated in healthy volunteers and cancer patients undergoing immunotherapy. In these studies, prominent tracer uptake in the heart was observed in both groups. Interestingly however, the signal observed in the heart wall of healthy volunteers was different than in cancer patients and patients undergoing immune modulating and other therapies. This discovery of different [¹⁸F]F—AraG signal profiles on healthy versus patients treated with therapeutic agents is harnessed in methods disclosed herein, including methods designed, for example, to use [¹⁸F]F—AraG ability to image the heart in order to monitor changes to heart cells caused by drugs or injury.

The use of [¹⁸F]F—AraG as a PET tracer in the methods disclosed herein offers significant advantages over conventional ¹⁸F labeled tracers in a number of distinct applications including observations of selected physiological phenomena such as myocardial perfusion, myocardial viability, and heart inflammation. In addition, as discussed below, methods of the invention include those designed to use [¹⁸F]F—AraG to observe patient physiological responses to various therapeutic agents, as well as using [¹⁸F]F—AraG as a PET tracer in drug development studies.

The invention disclosed herein has a number of embodiments. These embodiments include methods of imaging heart cells in a subject by administering to the subject a compound having a formula:

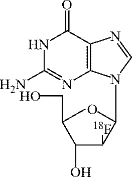 [SMILES: CC(CO)C(O)C([18F])C(O)n1cnc2c(=O)[nH]c(N)nc21]

wherein a route of administration is selected so as to allow the compound to be phosphorylated by deoxycytosine kinase and deoxyguanosine kinase present in heart cells in the subject; and then imaging the subject, wherein detecting the presence of the compound corresponds to the presence of heart cells. In some embodiments, these methods further comprise the step of using one or more images of the heart to assess one ormore parameters of myocardial perfusion in the heart of the subject. In some embodiments, these methods further comprise the step of using one or more images of the heart to assess one or more parameters of myocardial viability in the heart of the subject. In some embodiments, these methods further comprise the step of using one or more images of the heart to assess one or more parameters of inflammation in the heart of the subject. In certain embodiments of the invention, the subject has been administered a therapeutic agent, and one or more images of the heart are used to obtain information on the effect of the agent on the heart of the subject. Optionally, the therapeutic agent used in these methods is one that acts on mitochondria in the cells of the heart.

Embodiments of the invention also include methods of imaging selected cell populations (e.g., heart cells and leukocytes) in a patient suffering a pathological condition who is being treated one or more therapeutic agents. Such methods can be used to observe physiological changes in vivo that result from administration of such therapeutic agents (e.g., cardiotoxicity). Typically, such methods include administering to the subject undergoing treatment with the therapeutic agent(s) a PET probe compound having a formula:

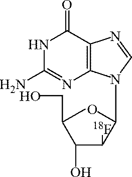 [SMILES: CC(CO)C(O)C([18F])C(O)n1cnc2c(=O)[nH]c(N)nc21]

wherein a route of administration is selected so as to allow the compound to be phosphorylated by deoxycytosine kinase and deoxyguanosine kinase present in cells in the subject. These methods further comprise PET imaging the subject, wherein detecting the presence of the PET probe compound corresponds to the presence of cells; and finallycorrelating the observed presence of the compound in cells of the subject with the subject’s response to the therapeutic agent. In certain embodiments of these methods, the subject is selected to be a patient diagnosed with cardiovascular disease; and/or the subject is selected to be a patient diagnosed with cancer; and/or the subject is selected to be a patient undergoing treatment for cardiovascular disease or cancer. In illustrative embodiments of the invention that are disclosed herein, the therapeutic agent is an anthracycline (e.g. doxorubicin, daunorubicin, epirubicin, idarubicin and the like), or an immune checkpoint inhibitor (e.g., immune checkpoint inhibitor selected to affect CTLA-4 or a PD-1 /PD-L1 blockade) and the and one or more images of the heart are used to obtain information on a physiological phenomena (e.g., cardiotoxicity) that results from the administration of the anthracycline or the immune checkpoint inhibitor. In illustrative working embodiments, the methods include observations of presence of the PET probe compound in heart cells, immune cells and/or in lymph nodes (e.g. tumor draining lymph nodes).

In certain embodiments of the invention, the PET methods of imaging cells in a subject responding to administration of a therapeutic agent further comprise observing one or more images obtained on a first date; observing one or more images on a second date; and then comparing the images obtained on the first imaging date with the images obtained on the second imaging date so as to observe changes in patient physiology over time that result from administration of the therapeutic agent (e.g. so as to distinguish between responders and nonresponders to the therapeutic agent). In certain embodiments of the invention, an amount of time from the first date to the second date comprises less than one week. Alternatively, an amount of time from the first date to the second date comprises at least one, two or three weeks, or at least one, two or three months.

Other embodiments of the invention include methods of imaging mitochondrial activity in cells in a subject by administering to the subject a compound having a formula:

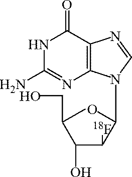 [SMILES: CC(CO)C(O)C([18F])C(O)n1cnc2c(=O)[nH]c(N)nc21]

wherein a route of administration is selected so as to allow the compound to be phosphorylated by deoxycytosine kinase and deoxyguanosine kinase present in cells in the subject; and then imaging the subject, wherein detecting the presence of the compound corresponds to the presence of mitochondrial activity. In certain embodiments, the subject is one identified as suffering from a mitochondrial deficient disease. In other embodiments of the invention the methods are part of a protocol designed to screen the subject for mitochondrial dysfunction; for example, the mitochondrial dysfunction associated with a cardiovascular disease, a neuropsychiatric disease, or a neurodegenerative disease. Optionally, the mitochondrial dysfunction is selected from the group consisting of myocardial perfusion, bipolar disorder, depression, schizophrenia Alzheimer’s disease, Parkinson’s disease, Friedreich’s ataxia, amyotrophic lateral sclerosis, Huntington’s disease, premature ageing, cardiomyopathy, a respiratory chain disorder, mtDNA depletion syndrome, myoclonus epilepsy, ragged-red fibers syndrome, myopathy encephalopathy lactic acidosis, stroke-like episodes, and optic atrophy.

Other objects features and advantages of the present invention will become apparent to those skilled in the art from the following detailed description. It is to be understood, however, that the detailed description and specific examples, while indicating some embodiments of the present invention are given by way of illustration and not limitation. Many changes and modifications within the scope of the present invention may be made without departing from the spirit thereof, and the invention includes all such modifications.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1.1A illustrates a number of synthesis schemes for making compounds of the present disclosure.

FIG. 1.1B illustrates a number of synthesis schemes for making compounds of the present disclosure.

FIG. 1.2 illustrates embodiments of R and R′.

FIG. 2.1 illustrates schematics of a synthesis of a [¹⁸F]F—AraG precursor and [¹⁸F]F—AraG.

FIG. 2.2 illustrates a graph of 5 × 10⁵CCRF-CEM cells were labeled with ¹⁸F-AraG (0.6 mCi/ml) for indicated times and doses. Uptake was measured on gamma counter. Bars represent mean of triplicate determinations +/- SEM.

FIG. 2.3A illustrates a graph of 5 × 10⁵CCRF-CEM cells were incubated, in triplicate, with 1uCi 8H³-AraG (1mCi/ml) and increasing amounts of cold 2F-AraG or DMSO for either 60 or 120 minutes. Percent of control uptake was calculated as: measured cpm of sample/ cpm accumulated 1uCi 8[H³]-AraG control x 100.

FIG. 2.3B illustrates a graph of 5 × 10⁵CCRF-CEM, MOLT-4 or RAJI cells, in triplicate, were labeled with 1 uCi 8-³H-AraG (1mCi/ml) in the presence of 1uM cold 2F-AraG, DMSO or media for 120 minutes. Percent of control uptake was calculated as in FIG. 2.3a.

FIG. 2.4 illustrates a schematic of the metabolism of 2′-deoxyguanosine (dGuo) by T lymphoblasts. In contrast to 2′-dGuo, AraG does not require ribonucleotide reductase activity for incorporation into DNA and is directly phosphorylated by mitochondrial dGK at low intracellular concentrations. At higher concentrations, it may also be phosphorylated by deoxycytidine kinase and incorporated into nuclear DNA. (Figure is from J Biol Chem. 2008;283:16437-16445).

FIG. 3.1 illustrates the analytical HPLC profile of co-injection of [¹⁸F]F-AraG with cold F—AraG standard (5% acetonitrile : 95% water; 1 mL/min, 254 nm, Phenomenex Gemini C18, 5 µ, 4.6 × 250 mm).

FIG. 3.2 illustrate a graph that shows 5 × 10⁵ CCRF-CEM cells, in triplicate, that were exposed to either 3 µCi or 10 µCi of [¹⁸F]F—AraG for 60 or 120 minutes. Cells took up ≃ 2-fold more [¹⁸F]F—AraG when exposed to 10 µCi, at 60 minutes (p=0.008) and at 120 minutes (p= 0.001) as compared to 3 µCi . Error bars represent S.E.M.

FIG. 3.3 illustrates a graph that shows 1 × 10⁶ purified primary T cells, stimulated with 100U/ mL IL.2, 50 nM PMA and 1 µg/mL, ionomycin, or un-stimulated, were incubated for 60 minutes with 1µCi of [¹⁸F]F—AraG. Error bars represent mean of triplicate determinations +/- SEM., n = 4 p=0.14 and 0.003 by two tailed, paired Student T test respectively.

FIG. 3.4 is a schematic diagram of scheme 1 that describes the synthesis of 2—N—Acetyl-6-O-((4-nitrophenyl)ethyl)-9-(3,5-di—O— trityl-2-trifyl-β-D-ribofuranosyl)guanine (2), the [¹⁸F]F—AraG precursor.

FIG. 3.5 is a schematic diagram of scheme 2 that describes the synthesis of 2′-Deoxy-2′-fluoro-9-β-D-arabinofuranosylguanine 5 (F—AraG).

FIG. 3.6 is a schematic diagram of scheme 3 that describes the synthesis of 2′-deoxy-2′-[¹⁸F]fluoro-9-β-D-arabinofuranosylguanine 7 ([¹⁸F]F—AraG).

FIG. 4 shows a cartoon schematic of a mechanism of imaging heart cells with [¹⁸F]F—AraG. [¹⁸F]F—AraG is transported into cells via nucleoside transporters, followed by the [18F]phosphorylation by mitochondrial deoxyguanosine kinase (dGK) and to a lesser extent by cytosolic deoxycytidine kinase (dCK). Phosphorylation leads to entrapment of [¹⁸F]F—AraG and allows visualization of these cells via PET imaging.

FIG. 5 provides data showing the kinetic properties of dCK and dGK for [³H]F—AraG. (a) Enzyme kinetics of dGK or dCK for [³H]F—AraG (b) dGuo positive control for dGK activity (c) dCyd positive control for dCK activity. Lines indicate best fit. The Km of dGK for [[³H]F—AraG was found to be 7.27 µM with a Vmax at 23.44 nmol/min/mg, while dCK had lower affinity for [³H]F—AraG (Km═50.89 µM, Vmax= 262.5). We observed high affinity of dCK for dCyd (Km=1.997, Vmax= 9.454) and similarly higher affinity in the dGuo positive control for dGK activity (Km═ 5.083, Vmax= 123.2).

FIG. 6 shows images of F—AraG signal in the heart in cancer patients. High mitochondrial activity in the heart results in high F—AraG signal one hour post injection of the tracer.

FIG. 7 provides data showing the intensity of the F—AraG signal in the heart wall of healthy volunteers and patients undergoing immunotherapy (pre refers to a pre-treatment image and post to an image taken 2-3 weeks after one injection of anti PD-1 antibody).

FIG. 8 provides a cartoon schematic showing mechanisms of imaging with [18F]F—AraG. [18F]F—AraG is transported into cells via nucleoside transporters, followed by the rate-limiting phosphorylation by mitochondrial deoxyguanosine kinase (dGK). Phosphorylation by dGK leads to entrapment and potential downstream accumulation into mtDNA allowing visualization via PET imaging.

FIG. 9 provides a cartoon schematic showing mechanisms of cardiotoxicity and its relation to imaging with [18F]F—AraG. A. Immune Checkpoint Inhibitor therapy (ICI)-induced Inflammation. Briefly, cancer therapies called immune checkpoint inhibitors (ICI) work by activating a patient’s own immune system to fight their cancer. ICI drugs that disrupt PD-1 and CTLA-4 signaling interfere with cardioprotective immunosuppressive mechanisms and lead to exaggerated proliferation and activation of immune cells. Excessive inflammatory activity can be visualized by [18F]F—AraG′s accumulation in activated T cells. B. Doxorubicin-induced mitochondrial damage. Doxorubicin readily enters mitochondria and interferes with mtDNA synthesis. Following phosphorylation by dGK, [18F]F—AraG can be incorporated into mtDNA and thus report on the status of its synthesis.

FIG. 10 provides maximum intensity projection (MIP) [18F]F—AraG images of a healthy subject (left) and a head and neck cancer patient. In some cancer patients, myocardial uptake was significantly higher than in healthy subjects, indicating aberrant mitochondrial/inflammatory activity.

FIGS. 11A-11B show data from the evaluation of [3H]F—AraG in human immune cells. FIG. 11A. The highest tracer uptake was observed in activated T cells. Macrophages (M1 and M2) and dendritic cells (DC) also showed accumulation. No significant accumulation was found in B cells, eosinophils, and neutrophils FIG. 11B. Activation of all subtypes of T cells led to an increase in tracer uptake, but activated CD8+ cells showed the highest increase.

FIGS. 12A-12C show data from embodiments of the invention. FIG. 12A. Tumor infiltrating lymphocytes take up more than 80% of tumor associated [18F]F—AraG, with CD8+ and CD4+ cells acquiring the largest portion of the tracer (72%). FACS analyses of the isolated lymphocytes showed an activated phenotype (CD44+ CD62L-) in majority of CD8+ (79.9±11.5) and CD4+ cells (88.3±11.7) FIG. 12B. [18F]F-AraG signal in the tumor ( white circles) and tumor draining lymph nodes (red) in mice pre- and 48 hours post single anti- PD-1 treatment. The responding mouse (R) showed a higher [18F]F—AraG signal both in tumor and tumor draining lymph nodes compared to the non-responding mouse (NR). FIG. 12C. The combination, intratumoral and intranodal, [18F]F—AraG signal in the responders was significantly higher (6.587 ± 0.6874, n=4) than in the non-responders (2.604 ± 1.083, n=4).

FIGS. 13A-13D show data from embodiments of the invention. FIG. 13A. Paclitaxel/carboplatin treatment, reported to cause immunologically silent death, did not lead to appreciable changes in [18F]F—AraG signal intensity. FIG. 13B. Dramatic increase in signal intensity was detected after oxaliplatin/cyclophosphamide treatment shown to induce immunogenic cell death. White circles indicate tumor draining lymph nodes, yellow arrows point to tumors. FIG. 13C. The [18F]F—AraG signal detected post oxaliplatin/cyclophosphamide treatment was significantly different than the pre therapy signal as well as the signal post paclitaxel-carboplatin treatment FIG. 13D. The ratio of CD8+ (effector) to CD4+FOXP3+ (regulatory) cells in the oxaliplatin/cyclophosphamide group was 27 times higher than in the paclitaxel/carboplatin treated mice, indicating immune active tumor microenvironment) (n=4 for each group).

FIGS. 14A-14D show data from studies of Ctla4+/- Pdcdl-/- mice present with cardiac immune infiltration. FIG. 14A. H&E images of lymphocytic infiltration in Ctla4+/- Pdcdl-/- mouse (left) and human (right; autopsy sample from myocardium of a patient that had complete heart block and ventricular tachycardia following ICI treatment.) FIG. 14B. Quantification of lymphoid infiltrate scores from H&E stained heart tissue and frequency of CD3, CD4, and CD8+ cells as a fraction of total nucleated cells. FIG. 14C. Representative images of CD3, CD4, and CD8 immunohistochemistry (right) stained heart tissue sections from female Ctla4+/- Pdcdl-/- mice. Left panels: IHC; Middle panels: segmentation with and blue=negative cells, right panels: positive cell density (blue=low, green=intermediate, yellow=high). Heatmap values represent arbitrary density units. FIG. 14D. Representative images of additional immunohistochemistry (CD3, F4/80+ macrophages and Foxp3+ Tregs) stained heart tissue from Ctla4+/- Pdcdl-/- mice.

FIGS. 15A-15C shows an outline of proposed studies of longitudinal [18F]F AraG monitoring of cardiotoxicity. FIG. 15A. Doxorubicin toxicity. Animals will be imaged before and 48 hours post weekly doxorubicin treatment. FIG. 15B. ICI toxicity. Animals will be imaged once a week starting in week 6. Preliminary studies showed week 6-8 to be the peak of myocardial immune infiltration. Animals will be followed until week 10. FIG. 15C. Dox/immunotherapy toxicity. Mice will be treated twice a week for two weeks. [18F]F AraG imaging will be performed 48 hours post 2nd and 4th treatment. Ex vivo analysis will be performed one day after the final scan.

FIG. 16 provides maximum intensity projection (MIP) [18F]F—AraG images of 18FDG and [18F]F—AraG myocardial uptake in a head and neck patient. The myocardial uptake in the diagnostic 18FDG obtained after fasting was low. The [18F]F—AraG cardiac uptake in the same patient showed higher uptake than in healthy volunteers (see FIG. 7).

FIG. 17 shows images from a comparison of 18FDG and [18F]F—AraG uptake in rat myocardium. The rats were imaged with tracers on consecutive days. While [18F]F—AraG showed reproducible myocardial uptake, 18FDG showed variation that can interfere with its clinical utility in myocardial imaging.

FIG. 18 shows images of myocardial [18F]F—AraG uptake in a healthy female volunteer taken at indicated time points post injection of the tracer. The cardiac signal persisted during the imaging window of 120 minutes and was uniform in nature.

FIG. 19 shows data on radiomics features extracted from [18F]F—AraG images of tumor bearing mice treated with anti-CTLA-4 and anti-PD-1 antibodies. Energy, entropy and uniformity were found to be significantly different between treated and untreated animals.

FIGS. 20A-20C show transverse [18F]F AraG PET images in a healthy volunteer in FIG. 20A and in a head and neck cancer patient in FIG. 20B. The signal in the cancer patient was significantly higher, with areas of focal increases (red arrows). FIG. 20C provides data showing that [18F]F AraG signal in the heart wall of cancer patients was significantly higher than in healthy subjects, both before and after immunotherapy. The increased signal before immunotherapy may indicate cardiotoxicity of prior anticancer treatments.

FIG. 21 shows data from electrocardiogram and [18F]F AraG images of three head and neck patients. The top patient shows relatively low and uniform [18F]F AraG myocardial uptake and a normal ECG. The other two patients show abnormal ECGs and higher (middle) and heterogenous [18F]F AraG uptake. The data in this figure illustrates key aspects of embodiments of the invention as it shows the correlation between tracer uptake and hear abnormalities as shown in the electrocardiograms

FIG. 22 provides maximum intensity projection (MIP) Images showing the change in myocardial signals in a patient with recurrent melanoma before and after immunotherapy infusion. Higher-than-normal myocardial uptake in the pretreatment scan may indicate cardiotoxicity of prior anticancer treatments. Only one immunotherapy infusion led to a dramatically increased myocardial uptake. Increased [18F]F—AraG signal was also observed in the thyroid and the spleen. The data in this figure illustrates key aspects of embodiments of the invention as it illustrates how [18F]F AraG can be used to image the effects of ICI on the heart in PET methodologies.

### DETAILED DESCRIPTION

Before the present disclosure is described in greater detail, it is to be understood that this disclosure is not limited to particular embodiments described, and the embodiment of the invention as such may, of course, vary. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this disclosure belongs. All publications and patents cited in this specification are herein incorporated by reference as if each individual publication or patent were specifically and individually indicated to be incorporated by reference and are incorporated herein by reference to disclose and describe the methods and/or materials in connection with which the publications are cited. As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a compound” includes a plurality of compounds. In this specification and in the claims that follow, reference will be made to a number of terms that shall be defined to have the following meanings unless a contrary intention is apparent.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. Further, documents or references cited in this text (e.g. U.S. patent Publication Nos. 20150230762, 20150297760 and 20190054198), in a Reference List before the claims, or in the text itself; and each of these documents or references (“herein cited references”), as well as each document or reference cited in each of the herein-cited references (including any manufacturer’s specifications, instructions, etc.) are hereby expressly incorporated herein by reference.

In accordance with the present disclosure, “a detectably effective amount” of embodiments of the present disclosure is defined as an amount sufficient to yield an acceptable image using equipment that is available for clinical use. A detectably effective amount of the embodiments of the present disclosure may be given in one or more administrations. The detectably effective amount of embodiments of the present disclosure can vary according to factors such as the degree of susceptibility of the individual, the age, sex, and weight of the individual, idiosyncratic responses of the individual, the dosimetry, and the like. Detectably effective amounts of embodiments of the present disclosure can also vary according to instrument and film-related factors. Optimization of such factors may bel within the level of skill in the art.

The term “detectable” refers to the ability to detect a signal or presence of an embodiment of the present disclosure over a background signal. The term “detectable signal” or the phrases “detection of a labeled compound” or “detectable labeled compound” refers to the detection (directly or indirectly) of a labeled compound in a host or sample. The detection of a labeled compound refers to the ability to detect and distinguish the presence of a labeled compound in a host or sample from other background signals derived from the host or sample. In other words, there is a measurable and statistically significant difference (e.g., a statistically significant difference is enough of a difference to distinguish among the detectable signal and the background, such as about 0.1%, 1%, 3%, 5%, 10%, 15%, 20%, 25%, 30%, or 40% or more difference between the detectable signal and the background) between detectable signal and the background. Standards and/or calibration curves can be used to determine the relative intensity of the detectable signal and/or the background. The detectable signal can be generated from a small to large concentration of a labeled compound. In an embodiment, the detectable signal may need to be the sum of each of the individual labeled compound signals. In an embodiment, the detectable signal can be generated from a summation, an integration, or other mathematical process, formula, or algorithm. In an embodiment, the summation, the integration, or other mathematical process, formula, or algorithm can be used to process the detectable signal so that the detectable signal can be distinguished from background noise and the like.

As used herein, “agent”, “active agent”, or the like, can include a compound (e.g., labeled compound) of the present disclosure. The agent can be disposed in a composition or a pharmaceutical composition. As used herein, “pharmaceutical composition” refers to the combination of an active agent with a pharmaceutically acceptable carrier. As used herein, a “pharmaceutical composition” refers to a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, intravenous, buccal, rectal, parenteral, intraperitoneal, intradermal, intracheal, intramuscular, subcutaneous, inhalational and the like.

A “pharmaceutically acceptable excipient,” “pharmaceutically acceptable diluent,” “pharmaceutically acceptable carrier,” or “pharmaceutically acceptable adjuvant” means an excipient, diluent, carrier, and/or adjuvant that are useful in preparing a pharmaceutical composition that are generally safe, non-toxic and neither biologically nor otherwise undesirable, and include an excipient, diluent, carrier, and adjuvant that are acceptable for veterinary use and/or human pharmaceutical use. For compositions suitable for administration to humans, the term “excipient” is meant to include, but is not limited to, those ingredients described in Remington: The Science and Practice of Pharmacy, Lippincott Williams & Wilkins, 21st ed. (2006) the contents of which are incorporated by reference herein.

The term “unit dosage form,” as used herein, refers to physically discrete units suitable as unitary dosages for human and/or animal subjects, each unit containing a predetermined quantity of a compound calculated in an amount sufficient (e.g., weight of host, disease, severity of the disease, etc) to produce the desired effect in association with a pharmaceutically acceptable diluent, carrier or vehicle. The specifications for unit dosage forms depend on the particular compound employed, the route and frequency of administration, and the effect to be achieved, and the pharmacodynamics associated with each compound in the host.

The term “effective amount” as used herein refers to that amount of an embodiment of the present disclosure (which may be referred to as a labeled compound) being administered that can be used to image a cell such as a heart cell. By “administration” is meant introducing an embodiment of the present disclosure into a subject. Administration can include routes, such as, but not limited to, intravenous, oral, topical, subcutaneous, intraperitoneal, intraarterial, inhalation, vaginal, rectal, nasal, introduction into the cerebrospinal fluid, or instillation into body compartments can be used.

As used herein, the term “host” or “subject” includes humans, mammals (e.g., cats, dogs, horses, etc.), and other living animals. In particular, the host is a human subject. Typical hosts to which embodiments of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. Additionally, for in vitro applications, such as in vitro diagnostic and research applications, body fluids and cell samples of the above subjects will be suitable for use as a “sample”, such as mammalian (particularly primate such as human) blood, urine, or tissue samples, or blood, urine, or tissue samples of the animals mentioned for veterinary applications.

The invention disclosed herein has a number of embodiments. For example, embodiments of the invention include methods of imaging heart cells in a subject/patient. Such methods include administering to the subject a compound having a formula:

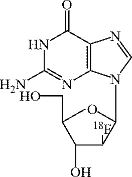 [SMILES: CC(CO)C(O)C([18F])C(O)n1cnc2c(=O)[nH]c(N)nc21]

wherein a route of administration is selected so as to allow the compound to be phosphorylated by deoxycytosine kinase and deoxyguanosine kinase present in heart cells in the subject; and then imaging the subject, wherein detecting the presence of the compound corresponds to the presence of heart cells. In certain embodiments of the invention, the subject is selected to be a patient that has been administered a therapeutic agent or a radiation therapy, and the one or more images of the heart obtained are used to obtain information on the effect of the agent or the radiation therapy on the heart of the subject. In illustrative embodiments of the invention that are disclosed herein, the therapeutic agent is an anthracycline such as doxorubicin or an immune checkpoint inhibitor and the and one or more images of the heart are used to obtain information on cardiotoxicity resulting from the administration of doxorubicin or the immune checkpoint inhibitor. In certain embodiments of the invention, the therapeutic agent is selected to be one observed to act on mitochondria in the cells of the heart.

In certain methods of the invention, the subject is selected to be a patient diagnosed with cardiovascular disease; and/or the subject is selected to be a patient diagnosed with cancer; and/or the subject is selected to be a patient undergoing treatment for cardiovascular disease or cancer. Optionally, the subject is a patient treated with at least one therapeutic agent comprising an immune checkpoint inhibitor selected to affect a CTLA-4 or a PD-1/PD-L1 blockade (e.g. an antibody such as pembrolizumab; nivolumab; atezolizumab; avelumab; bevacizumab; and durvalumab). In certain embodiments of the invention, the methods further comprise observing one or more images of the heart on a first date; observing one or more images of the heart on a second date; and then comparing the images obtained on the first date with the images obtained on the second date so as to observe changes in the heart over time. In some embodiments of the invention, an amount of time from the first date to the second date comprises less than one week. Alternatively in other embodiments of the invention, an amount of time from the first date to the second date comprises at least one, two or three weeks, or at least one, two or three months. Some embodiments of the invention include using one or more images of the heart obtained to: assess one or more parameters of myocardial perfusion in the heart of the subject; and/or assess one or more parameters of myocardial viability in the heart of the subject; and/or assess one or more parameters of inflammation in the heart of the subject.

Embodiments of the invention also include methods of imaging cells in a subject responding to administration of a therapeutic agent. Such methods can be used to observe physiological changes in vivo that result from administration of the therapeutic compound (e.g., cardiotoxicity, drug responsiveness and the like). Typically, such methods include administering the therapeutic agent to the subject, and (typically after a period of time such as at least 48 hours, at least 1 week or at least one month) then administering to the subject a PET probe compound having a formula:

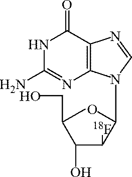 [SMILES: CC(CO)C(O)C([18F])C(O)n1cnc2c(=O)[nH]c(N)nc21]

wherein a route of administration is selected so as to allow the compound to be phosphorylated by deoxycytosine kinase and deoxyguanosine kinase present in cells in the subject (e.g. heart cells, cancer cells or leukocytes). These methods further comprise PET imaging the subject, wherein detecting the presence of the PET probe compound corresponds to the presence of cells; and finally correlating the observed presence of the compound in cells of the subject with the subject’s response to the therapeutic agent. In some embodiments of the invention, such correlation steps include comparisons to a control such as typical [¹⁸F]F-AraG PET image profiles in healthy humans), [¹⁸F]F-AraG PET images obtained from the patient prior to administration of the agent or the like. In certain working embodiments of such methods that are disclosed herein, the correlating step includes observations of presence of the PET probe compound in heart cells, immune cells and/or in lymph nodes (e.g. tumor draining lymph nodes). In certain embodiments of these methods, the subject is selected to be a patient diagnosed with cardiovascular disease; and/or the subject is selected to be a patient diagnosed with cancer; and/or the subject is selected to be a patient undergoing treatment for cardiovascular disease or cancer. In illustrative embodiments of the invention that are disclosed herein, the therapeutic agent is an agent observed to modulate mitochondrial physiology, an anthracycline such as doxorubicin or an immune checkpoint inhibitor (e.g., immune checkpoint inhibitor selected to affect CTLA-4 or a PD-1:PD-L1 blockade) and the and one or more images of the heart are used to obtain information on cardiotoxicity resulting from the administration of doxorubicin or the immune checkpoint inhibitor.

In certain embodiments of the invention, the methods of imaging cells in a subject responding to administration of a therapeutic agent further comprise observing one or more images obtained on a first date/time; observing one or more images at a second date/time; and then comparing the images obtained on the first date with the images obtained on the second date so as to observe changes in patient physiology over time that result from administration of the therapeutic agent (e.g. so as to distinguish between responders and nonresponders to the therapeutic agent). In certain embodiments of the invention, an amount of time from the first date to the second date comprises less than one week. Alternatively an amount of time from the first date to the second date comprises at least one, two or three weeks, or at least one, two or three months.

Yet another embodiment of the invention is a method of imaging mitochondrial activity in cells in a subject. Typically these methods include administering to the subject a compound having a formula:

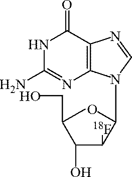 [SMILES: CC(CO)C(O)C([18F])C(O)n1cnc2c(=O)[nH]c(N)nc21]

wherein a route of administration is selected so as to allow the compound to be phosphorylated by deoxycytosine kinase and deoxyguanosine kinase present in cells in the subject; and then imaging the subject, wherein detecting the presence of the compound corresponds to the presence of mitochondrial activity such that mitochondrial activity in cells in a subject is observed. Typically in these methods, the subject is a patient selected as suffering from a mitochondrial deficient disease. In certain embodiments, the method is used to screen the subject for mitochondrial dysfunction; and the mitochondrial dysfunction is a cardiovascular disease, a neuropsychiatric disease, or a neurodegenerative disease. In some embodiments, the method is used to screen the subject for a mitochondrial dysfunction selected from the group consisting of myocardial perfusion, bipolar disorder, depression, schizophrenia Alzheimer’s disease, Parkinson’s disease, Friedreich’s ataxia, amyotrophic lateral sclerosis, Huntington’s disease, prematureageing, cardiomyopathy, a respiratory chain disorder, mtDNA depletion syndrome, myoclonus epilepsy, ragged-red fibers syndrome, myopathy encephalopathy lactic acidosis, stroke-like episodes, and optic atrophy.

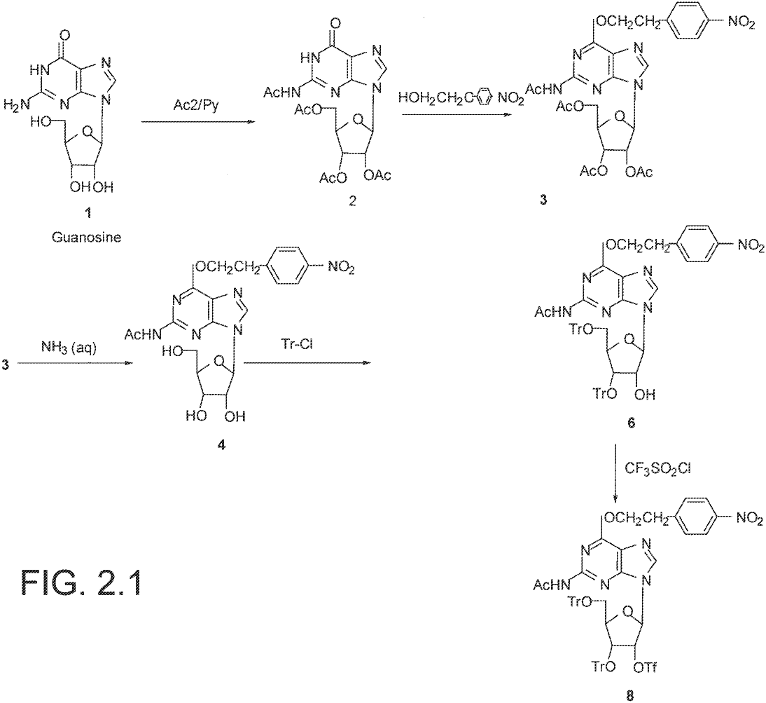

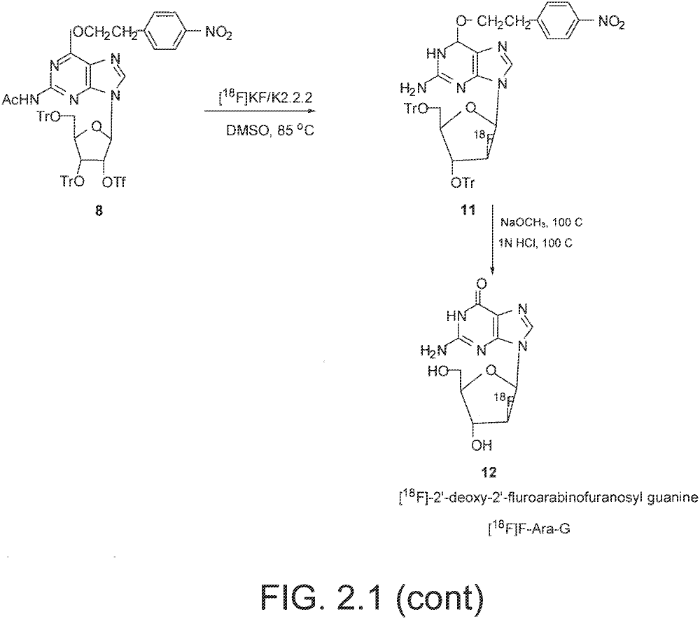

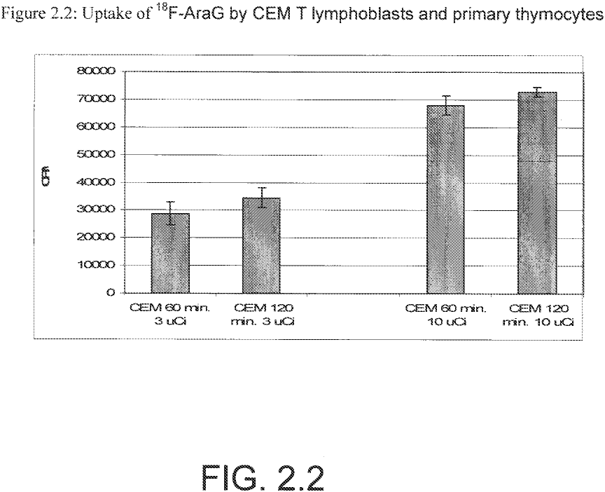

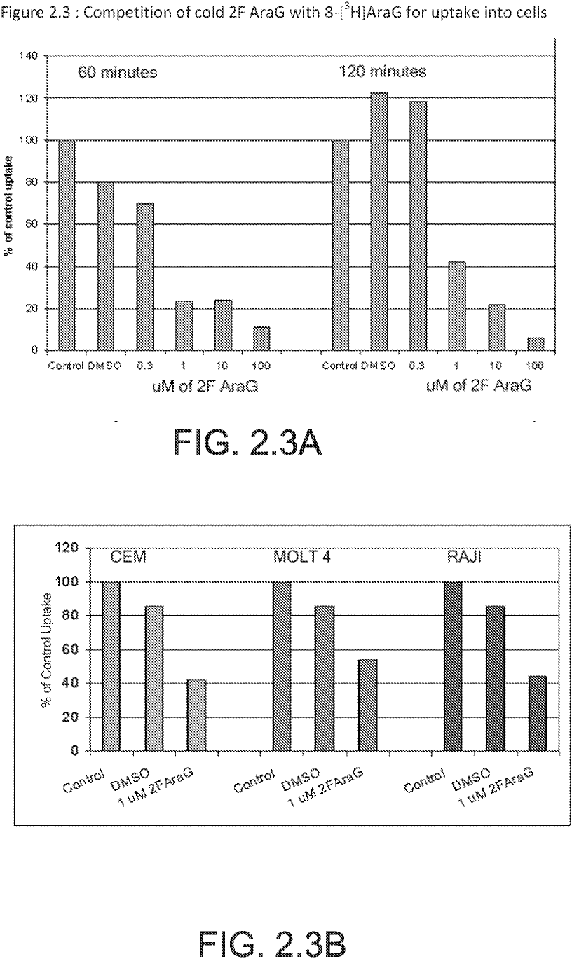

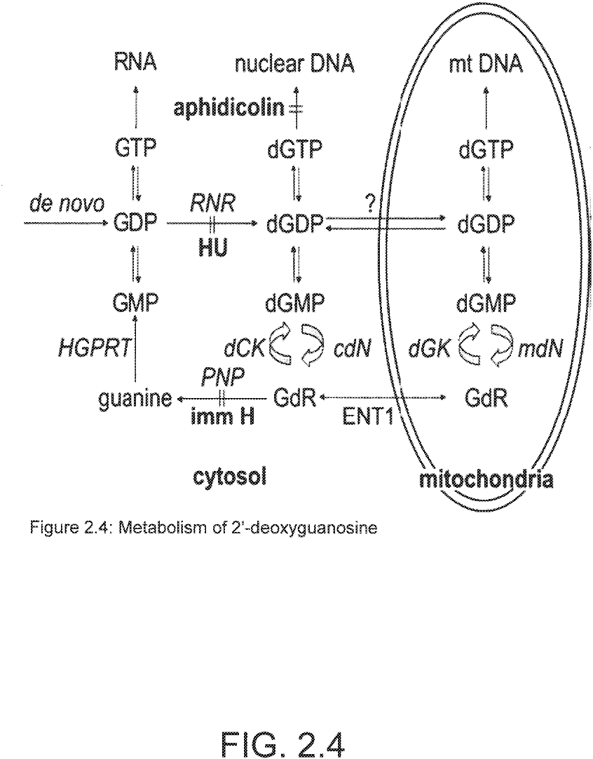

Briefly, in vivo AraG is metabolized in a unique fashion by deoxyguanosine kinase and incorporated into mitochondrial DNA (FIG. 2.4). These observations led to the synthesis of a more water-soluble AraG prodrug, 2-amino-6-methoxypurine arabinoside (506U, Nelarabine), for potential clinical application in the treatment of T lymphoblastic diseases. This compound, developed over a number of years by Glaxo, is now FDA-approved for the treatment of relapsed T cell ALL and T cell lymphoblastic lymphomas.

Embodiments of the present disclosure include methods of using [18F]—F—arabinofuranosyl guanine compounds, compounds that can be formed by a number of processes such as those disclosed in U.S. Pat. Application Serial No. 17/314,366 Filed on May 7, 2021 and entitled: METHODS AND MATERIALS FOR MAKING PET RADIOTRACERS, the contents of which are incorporated herein by reference. Such embodiments of the invention include, for example, [18F]—F—arabinofuranosyl guanine compounds formed from compositions of matter comprising a compound having the general formula:

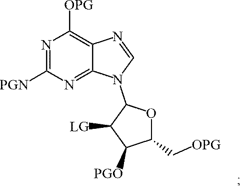

wherein: PG comprises a protecting group; and LG comprises a leaving group. Typically in such embodiments of the invention, the nitrogen atom coupled to the protecting group is coupled to two protecting groups as would be represented by “N(PG)₂”. Alternatively, this nitrogen atom is coupled to a hydrogen atom and a single protecting group as would be represented by “NHPG”. In illustrative embodiments of the invention, the composition includes at least one of precursor 1 or precursor 3:

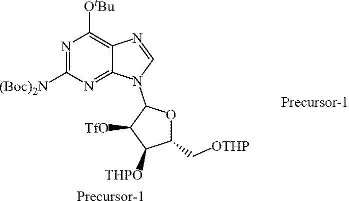

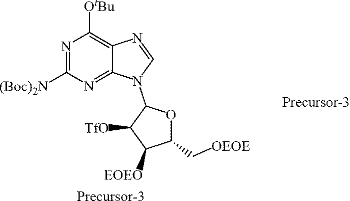

wherein: ^{t}Bu comprises a tert-Butyloxycarbonyl alcohol protecting group; Boc comprises a tert-Butyloxycarbonyl amine protecting group; THP comprises a tetrahydropyranyl alcohol protecting group; EOE comprises an ethoxyethyl alcohol protecting group; and Tf comprises a triflate leaving group.

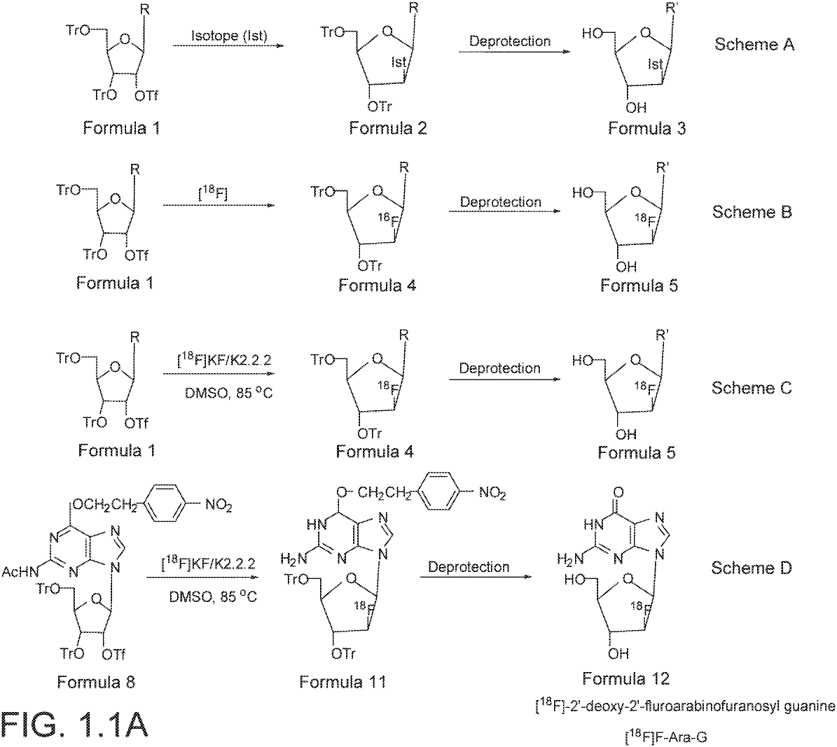

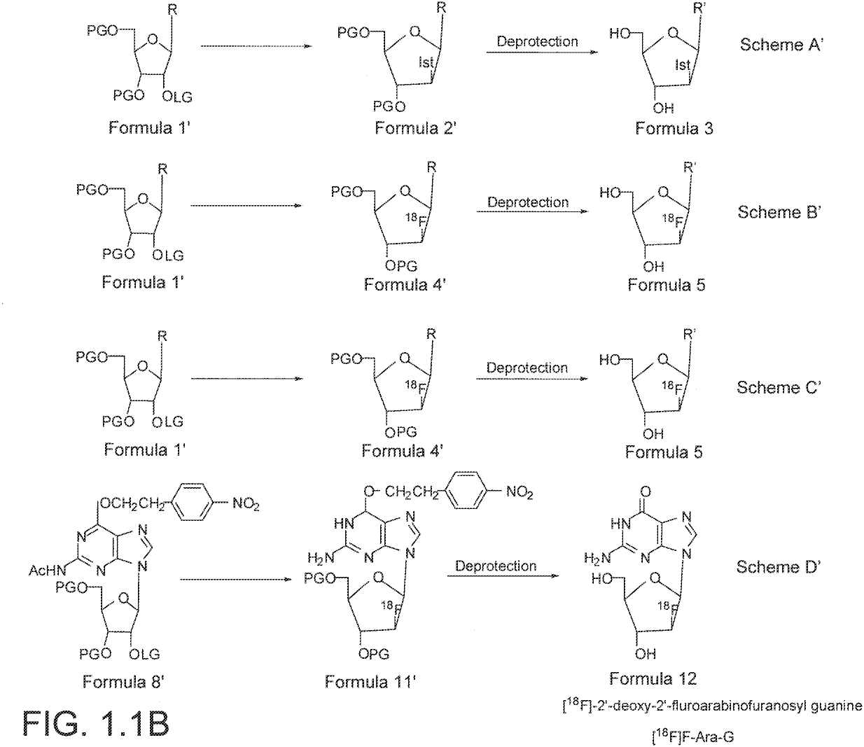

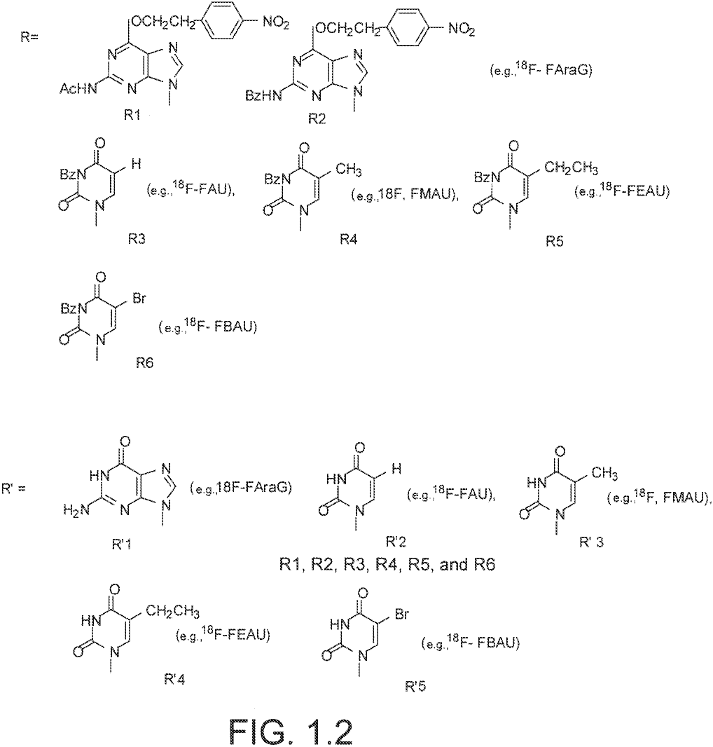

Embodiments of the present disclosure include methods of using [18F]—F—arabinofuranosyl guanine compounds formed by other processes such as those disclosed in U.S. Application entitled “Compounds and Methods of Making Compounds”, having serial number 17/185,502, filed on Feb. 25, 2021, the contents of which are incorporated herein by reference. Such methods include compounds such as those shown in FIGS. 1.1A and 1.1B having formula 2, 3, 4, 5, 11, and 12 and formula 2′, 3′, 4′, 5′, and 11′, as well as uses for the compounds for imaging, and the like.

An illustrative embodiment of making a labeled compound, among others, includes: reacting a compound including an isotope (Ist) with a compound having formula 1′,

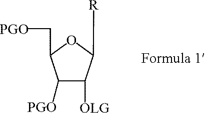

to form a compound having formula 2′,

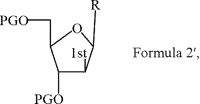

; and conducting deprotection on the compound having formula 2′ to form a compound having formula 3,

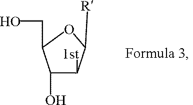

wherein PG is a protecting group and LG is a leaving group, and wherein R is a compound having a formula selected from the group consisting of R1, R2, R3, R4, R5, and R6:

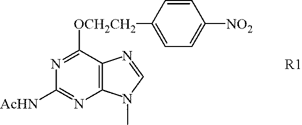

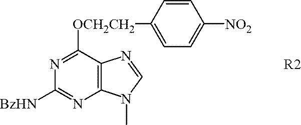

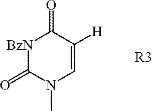

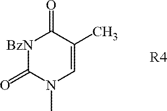

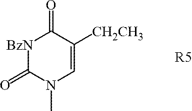

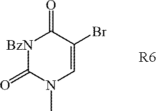

and wherein R′ is a compound having a formula selected from the group consisting of R′1, R′2, R′3,

R′4, and R′5, wherein Ac is an acetyl group, and Bz is a benzoyl group, where each of Ac and Bz can be replaced as described herein:

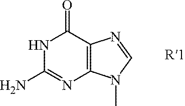

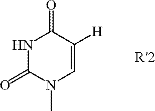

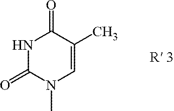

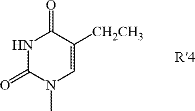

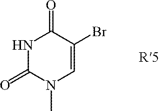

An illustrative embodiment of a labeled compound, among others, includes: a labeled compound, comprising: a compound having formula 3,

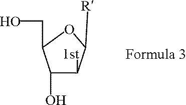

wherein Ist is an isotope, wherein R′ is a compound having a formula selected from the group consisting of R′1, R′2, R′3, R′4, and R′5:

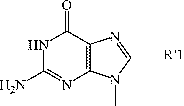

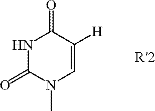

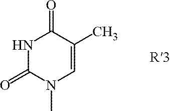

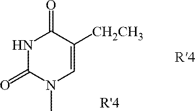

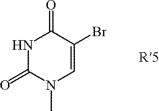

An illustrative embodiment of a labeled compound, among others, includes: a compound having formula 2′,

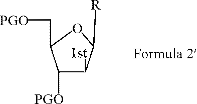

wherein PG is a protecting group, and wherein R is a group having a formula selected from the group consisting of R1, R2, R3, R4, R5, and R6:

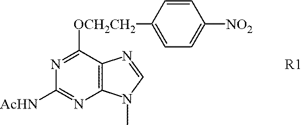

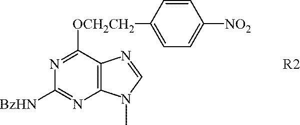

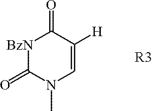

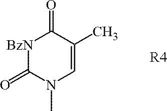

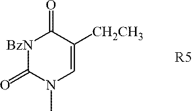

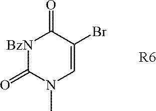

, and wherein Ac is an acetyl group, and Bz is a benzoyl group, where each of Ac and Bz can be replaced as described herein.

An illustrative embodiment of the method of imaging heart cells, among others, includes: administering to the subject a compound of the present disclosure; and imaging the subject, wherein detecting the presence of the compound corresponds to the presence of the heart cells. Another illustrative embodiment of the method of imaging the presence or extent of mitochondrial activity in cells, among others, includes: administering to the subject a compound of the present disclosure; and imaging the subject, wherein detecting the presence of the compound corresponds to the presence or extent of mitochondrial activity in cells in the subject.

In a demonstration of one working embodiment of the invention, the use of [¹⁸F]F—AraG as a PET tracer was evaluated in healthy volunteers as well as in cancer patients undergoing immunotherapy. In these studies, prominent tracer uptake in the heart was observed in both groups. Surprisingly however, when using this specific PET tracer, the signal observed in the heart wall of healthy volunteers was observed to be different in patients undergoing immunotherapy This unexpected discovery illustrates [¹⁸F]F-AraG’s ability to image changes to heart cells caused by drugs such as immunomodulatory agents, chemotherapeutic agents and the like, as well as other damage to the heart such as injury resulting from the exposure to radiation. In this context, because it can be used to evaluate mitochondrial activity, [¹⁸F]F—AraG can be used in methods for examining new agents that may improve mitochondrial function and/or assess effectiveness of the ability of these types of agents to improve mitochondrial function. In view of this discovery, the use [¹⁸F]F—AraG as a PET tracer addresses a need in this technology by providing significant advantages over the existing ¹⁸F labeled tracers in several distinct applications including observations of myocardial perfusion, myocardial viability and heart inflammation. In addition, the use [¹⁸F]F—AraG as a PET tracer can also be used in cardiac drug development studies as well as to observe the treatment responses to agents that modulate physiological (e.g. mitochondrial) activity (see, e.g. Zinovkin et al., Curr Mol Pharmacol. 2019;12(3):202-214).

As noted above, embodiments of the present disclosure include compounds such as those shown in FIGS. 1.1A and 1.1B having formula 2, 3, 4, 5, 11, and 12 and formula 2′, 4′, and 11′, as well as uses for the compounds for imaging, for example. Embodiments of the present disclosure are advantageous because the compounds can be made in a few simple steps, as described in detail below and in Examples 1 to 3. In particular, embodiments of the present disclosure provide for the direct fluorination of a precursor of a guanosine nucleoside followed by removal of a protecting group. Embodiments of the method include two steps and these two steps occur over a short period of time, both of which are advantageous relative to other possible alternative commercial production schemes.

Embodiments of the method are shown in FIGS. 1.1A and 1.1B in schemes A to D and schemes A′ to D′. Scheme A is generic, but uses specific protecting group (PG) and leaving group (LG), and schemes B to D provide additional details. Scheme A′ to D′ are generic in that they do not use specific protecting groups and leaving groups. It should be noted that the reagents can be changed in a manner similar to that described below. A more detailed scheme for an embodiment of the present disclosure is described in schemes 1 and 2 in Example 1 and Example 2. In general, embodiments of the method include making a labeled compound such as those embodied in formulae 3, 5, and 12. In an embodiment, the method can include reacting a compound including an isotope (Ist) with a compound having formula 1 in FIG. 1.1A to form a compound having formula 2 in FIG. 1.1A. The synthesis described in FIGS. 1.1A and 1.1B are very similar, with the primary difference being the use of specific PG and LG in FIG. 1.1A. So the following discussion about the synthesis in FIG. 1.1A can be applied to the synthesis in FIG. 1.1B. The various substitutions for protecting groups, leaving groups, reactants, and the like, described herein can be used in the synthesis described in FIG. 1.1B.

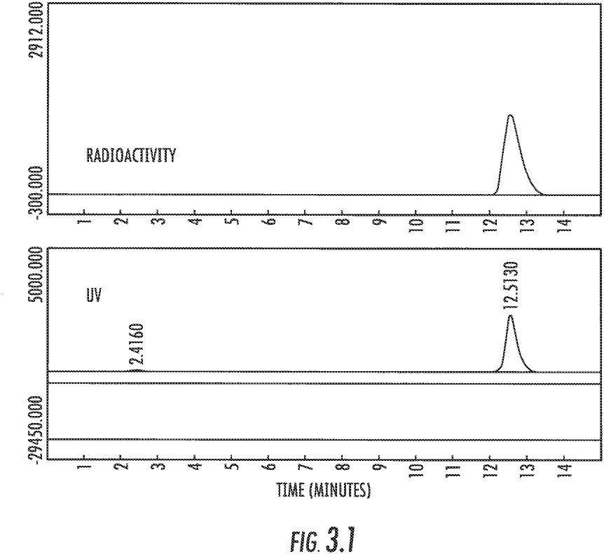

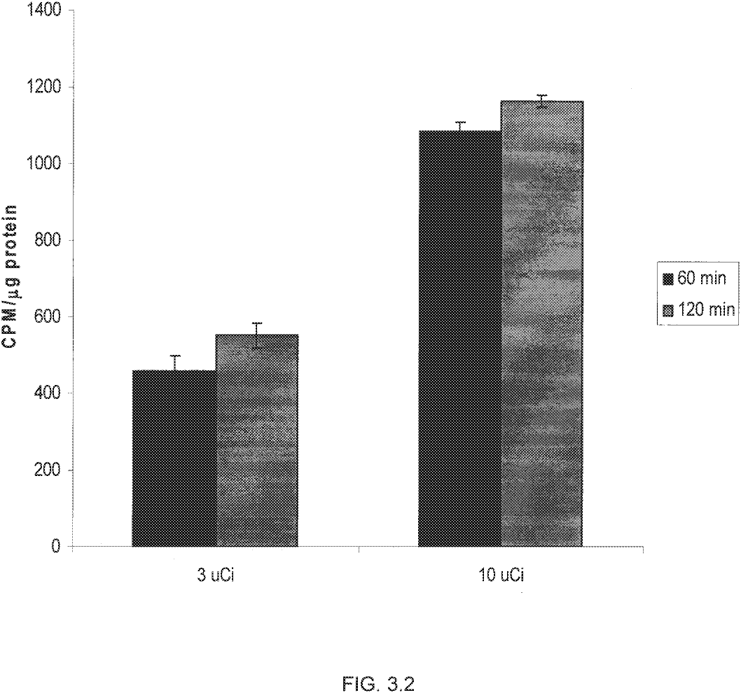

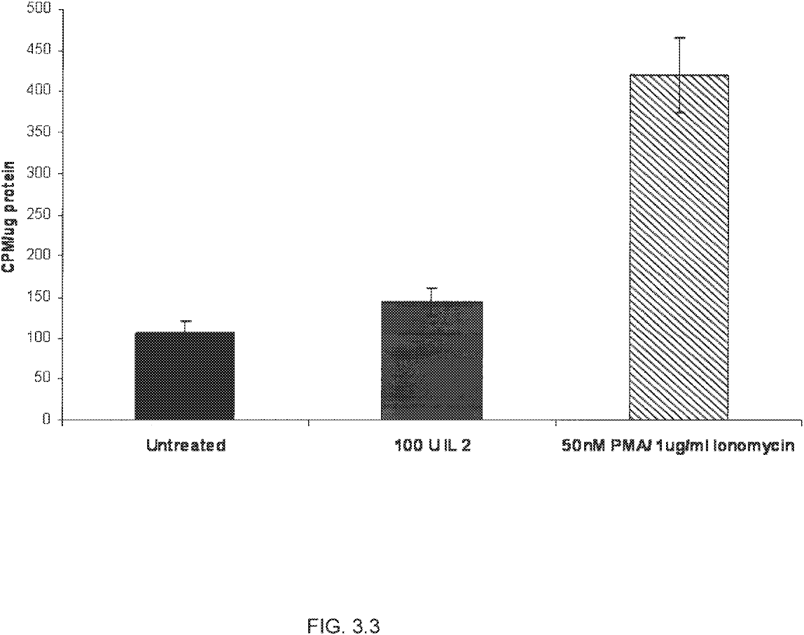

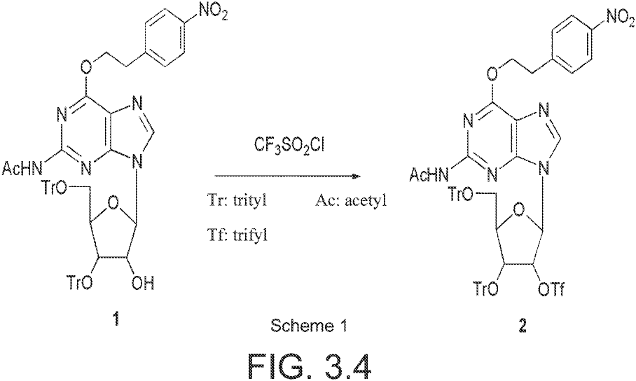

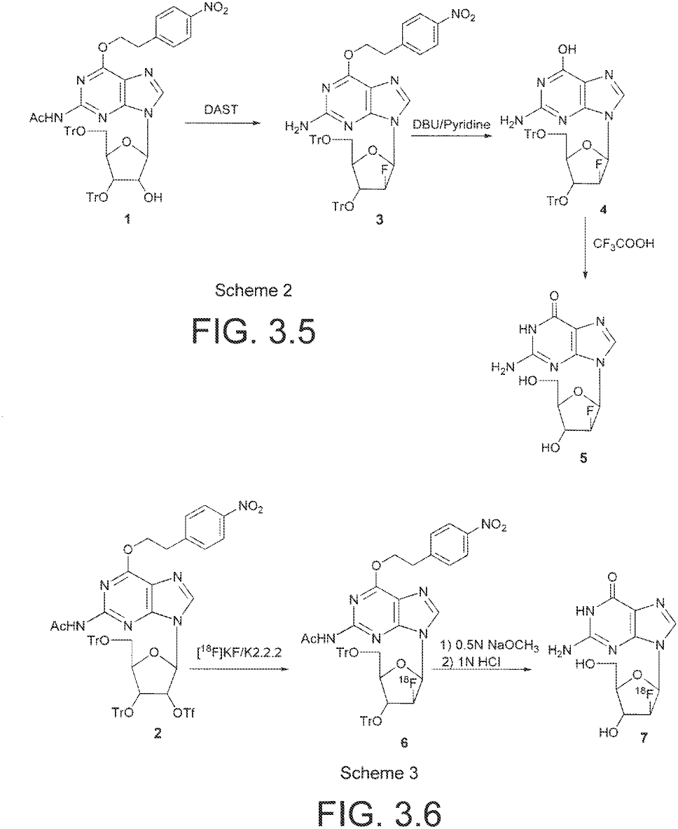

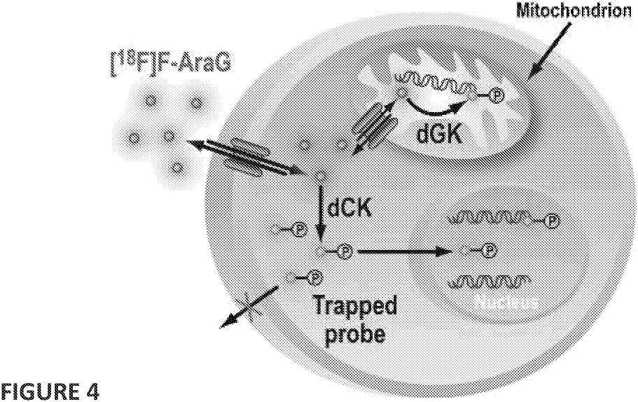

FIG. 2.1 describe schemes 1 and 2, which are for a specific embodiment of the present disclosure, and are described in detail in Example 1. In particular, the ¹⁸F(FAraG precursor (compound 8) is produced and then reacted to form ¹⁸F(FAraG precursor (compound 12). The details regarding the reaction steps are shown in FIG. 2.1, which are similar to those described above for the general synthesis. FIGS. 3.4 to 3.6 also provide specific details regarding the synthesis of embodiments of the present disclosure, and are described in detail in Example 2.

Embodiments of this disclosure also include methods of imaging heart tissues and cells. In general, embodiments of the labeled compounds can be used to image the localization and/or quantity of heart cells in subjects (e.g., a living human). The labeled compounds can be administered to the subject and then the subject or a portion of the subject can be imaged using a device such as Positron Emission Tomography (PET) to detect the presence and location within the subject, and/or quantity of the labeled compounds present The presence and/or quantity can be used to detect the presence, location, and/or number/size of heart cells, cancer cells and/or leukocytes in the subject.

The present disclosure can also provides packaged compositions including the precursor compounds or intermediates to the labeled compounds (e.g., formulae 1, 1′, 2, or 2′) and instructions for making the labeled compounds and methods of use (e.g., written instructions for their use). The kit can further include appropriate buffers and reagents known in the art for administering embodiments of the present disclosure to a subject.

### EXAMPLES

The following examples are put forth so as to provide those of ordinary skill in the art with a complete disclosure and description of how to make and use the present disclosure, and are not intended to limit the scope of what the inventors regard as their disclosure nor are they intended to represent that the experiments below are all or the only experiments performed.

### Example 1: Illustrative Methods for Making Pet Probes

AraG is a nucleoside analog that has proven efficacy in the treatment of T cell lymphoblastic diseases. It is metabolized in a unique fashion by deoxyguanosine kinase and incorporated into mitochondrial DNA. We have synthesized the ¹⁸F derivative to use as a molecular probe. Artisans can examine the uptake and metabolism in cell lines and determine the efficacy of this compound in imaging T lymphoblasts in a mouse model and activated T cells in a mouse model of acute graft versus host disease.

Previously we successfully synthesized a novel ¹⁸F derivative of 2-deoxyguanosine analog, 9-β-D-arabinofuranosylguanine (“¹⁸F-AraG” or “[¹⁸F]F—AraG)”, to use as a molecular probe (FIG. 2.1). [¹⁸F]AraG cell uptake was evaluated in leukemia cell line, CCRF-CEM, and was compared with [³H]AraG cell uptake. FIG. 2.2 shows uptake of [¹⁸F]AraG by CCRF-CEM cell and indicates that the uptake is dose dependent. To determine whether cold derivative 2F-AraG competes with the uptake of 8-[³H]—AraG, we studied the [³H]-Arag uptakes by CCRF-CEM, MOLT-4 (Leukemia cell line) and Raji (human Burkitt’s lymphoma cell line) and competition with cold 2F-AraG (FIGS. 2.3A and 2.3B). FIG. 2.3A shows that increasing the amount of 2F-AraG (1-100 µM) causes decrease of the [³H]—AraG uptake. Similar results were observed for MOLT4 and Raji cell lines (FIG. 2.3B). Competition assays indicate that cold derivative 2F-Arag competes with the uptake of the 8-[³H]—AraG, indicating similar uptake pathways.

Initial micro PET scans in normal nude mice indicate that ¹⁸F-AraG is taken up within lymph nodes. FIG. 2.1 describes an embodiment of a method of making compounds of the present disclosure. The 2′-deoxy-2′-fluoro-arabino nucleosides have been reported as antiviral agents (Proc Natl Acad Sci USA, 1992; 89: 2970-2974; Pharmacol 1999; 43: 233-240; J Pharm Chem 1996; 85: 339-344, each of which is incorporated herein by reference). We have been exploring the radio-labeled 8-[¹⁸F]fluoroguanine derivatives as potential in-vivo probes for imaging gene expression with Positron Emission Tomography (PET). We have developed a method for the preparation of 8-[¹⁸F]fluoroguanine derivatives based on a direct radiofluorination reaction and were able to synthesize 8-[¹⁸F]fluoroguanosine from guanosine (Nuclear Medicine and Biology. 2000; 27(2): 157-162, which is incorporated herein by reference). Recently, the synthesis of 2′-deoxy-2′- [¹⁸F]fluoro-9-β-D-arabinofuranosyladenine ([¹⁸]FAA] has been reported (J Label Comp Radiopharm 2003; 46: 805-814, which is incorporated herein by reference).

### Example 2: ^{[18}F]F-ARAG As A Pet Imaging Agent For Leukocytes

9-(β—D—Arabinofuranosyl)guanine (AraG) is a guanosine analog that has a proven efficacy in the treatment of T cell lymphoblastic disease. To test the possibility of using a radiofluorinated AraG as an imaging agent we have synthesized 2′-deoxy-2′-[¹⁸F]fluoro-9-(β-D-arabinofuranosylguanine ([¹⁸F]F—AraG) and investigated its uptake in T-Cells.

To this end, we have synthesized [¹⁸F]F—AraG via a direct fluorination of 2-N-acetyl-6-O-((4-nitrophenyl)ethyl)-9-(3′,5′-di-O-trityl-2′—O—trifyl-β-D-ribofuranosyl)guanine with [¹⁸F]KF/K.2.2.2 in DMSO at 85° C. for 45 minutes. [¹⁸F]F—AraG uptake in both a CCRF-CEM leukemia cell line (unactivated) and activated primary thymocytes was evaluated. We have successfully prepared [¹⁸F]F—AraG in 7-10% radiochemical yield (decay corrected) with a specific activity of 0.8-1.3 Ci/µmol. Preliminary cell uptake experiments showed that both a CCRF-CEM leukemia cell line and activated primary thymocytes take up the [¹⁸F]F—AraG. [¹⁸F]F—AraG has been successfully synthesized by direct fluorination of an appropriate precursor of a guanosine nucleoside. This approach could be used for the synthesis of other important PET probes such as [¹⁸F]FEAU, [¹⁸F]FMAU and [¹⁸F]FBAU which are currently synthesized by multiple steps and involve lengthy purification. The cell uptake studies support future studies to investigate the use of [¹⁸F]F—AraG as a PET imaging agent of T-cells.

Scheme 1, as shown in FIG. 3.4, shows the synthesis of 2—N—Acetyl-6-O-((4-nitrophenyl)ethyl)-9-(3,5-di-O-trityl-2-trifyl-β-D-ribiofuranosyl)guanine (2), the [¹⁸F]F—AraG precursor. Treatment of 2′,5′-di-O-trityl guanosine derivative 1 with CF₃SO₂Cl/DMAP afforded 2-N-acetyl-6-O-((4-nitrophenyl)ethyl)-9-(3′,5′-di-O-trityl-2′—O—trifyl-β-D-ribofuranosyl)guanine (2) in 65% yield. Scheme 2, as shown in FIG. 3.5, shows the synthesis of cold F—AraG standard which was prepared according to the literature procedure (J. Org. Chem. 1992, 57, 7315-7321, which is incorporated herein by reference). 3′, 5′—Di—O—trityl guanosine derivative 1 was converted to 6-O-((4-nitrophenyl)ethyl)-9-(3′,5′-di-O-trityl-2′-fluoro-p-D-arabinofuranosyl)guanine 3 with DAST reagent. De-protection of 3 with DBU afforded 9-(3′, 5′-di-O-trityl-2′-fluoro-β-D-arabinofuranosyl)guanine (4). Finally, de-protection of 4 by TFA afforded 2′-deoxy-2′-fluoro-9-β-D-arabinofuranosylguanine 5 (F—AraG).

### Radiochemistry:

[¹⁸F]-labeled guanosine derivative 6 (Scheme 3 as shown in FIG. 3.6) was prepared by nucleophilic displacement of triflate in 2 by [¹⁸F]fluoride ion in DMSO at 85° C. for 45 min. Purification of [¹⁸F]6 via HPLC was required to avoid contamination of the final product [¹⁸F]F—AraG 7 with the de-protected starting material 2 (AraG). [¹⁸F]6 was smoothly hydrolyzed first by base (0.5 M NaOCH₃) and then by acid (1N HCl) to yield [¹⁸F]F—AraG 7. The radiochemical yield was 7-10 % (decay corrected, n = 10) and the specific activity was 0.8-1.3 Ci /µmol. Analytical HPLC profile of co-injection of 7 with cold F—AraG standard is shown in FIG. 3.1.

To evaluate the ability of cells to uptake [¹⁸F]F—AraG, the CCRF-CEM cell line (acute lymphoblastic T leukemia cells, unactivated) and primary T-cells were exposed to [¹⁸F]F—AraG. FIG. 3.2 shows the uptake of [¹⁸F]F—AraG by unactivated CCRF-CEM cell and indicates that [¹⁸F]F—AraG uptake is dose dependent with a 2-fold (P<XX) increase in [¹⁸F]F—AraG uptake by cells exposed to 10 µCi compared to cells exposed to 3 µCi. We then looked to see if activated primary thymocytes derived from normal mouse tissue, would also accumulate [¹⁸F]F—AraG. FIG. 3.3 represents the data that primary T cells stimulated with 100U/mL of interleukin 2 take up 1.4-fold more (p=0.14) [¹⁸F] F—AraG and primary T cells stimulate with 50 nM PMA and 1 µg/mL ionomycin take up 4.7 fold more (p= 0.003) [¹⁸F] F—AraG than un-stimulated primary T cells.

There are many reports on the indirect synthesis of 2′-deoxy-2′-fluoro-9-β-D-arabinofuranosylguanine (F—AraG) in which fluorine is first introduced in the arabino position at C-2, and then fluorinated sugar reacted with the purine base (Carbohydr. Res. 1975, 42, 233-240, J. Org. Chem. 1985, 50, 3644-3647, and Chem. Pharm. Bull. 1989, 37, 336-339, each of which are incorporated herein by reference). However, there is only one report on the direct synthesis of cold F—AraG in which fluorine is incorporated into arabino position at C-2 of the sugar by direct fluorination of an appropriately protected guanosine derivative with DAST (J. Org. Chem. 1992, 57, 7315-7321, which is incorporated herein by reference). The synthesis of [¹⁸F]labeled F—AraG has not been reported to date. Due to the difficult synthesis of [¹⁸F]labeled DAST and long reaction times of DAST mediated fluorinations, it is not practical to synthesize [¹⁸F]F—AraG via the [¹⁸F]DAST method. Compound 2, the [¹⁸F]F—AraG precursor was prepared (scheme 1 as shown in FIG. 3.4) and characterized by ¹H and ¹⁹F NMR spectroscopy and high resolution mass spectrometry. Chemical shift of H2′ changed from 4.73 ppm in 1 to 6.03 ppm in 2 due to the electronegativity of the trifyl group at C2′ position. ¹⁹F NMR showed a singlet at -75.00 ppm which is consistent with the chemical shift of sugar triflates. Similar chemical shift trends were observed for the synthesis of adenosine triflate [19]. To the best of our knowledge precursor 2 is new and has been synthesized for the first time in our laboratory (provisional patent filed). Also, for the first time, we have synthesized [¹⁸F]F—AraG (7, scheme 3 as shown in FIG. 3.6) via a direct fluorination of 2 with [¹⁸F]KF/K.2.2.2 in DMSO in 7-10 % radiochemical yield (decay corrected) with a specific activity of 0.8-1.3 Ci/µmol. The identity and purity of 7 was confirmed by co-injection with an authentic standard compound 5 on an analytical HPLC column (FIG. 3.1).

To evaluate the performance of [¹⁸F]F—AraG 7 in cell culture, we performed several assays. To ascertain the ability of cells to uptake [¹⁸F]F—AraG we exposed the CCRF-CEM cell line (acute lymphoblastic T leukemia cells, unactivated) and primary T-cells to [¹⁸F]F—AraG. FIG. 3.2 shows the uptake of [¹⁸F]F—AraG by CCRF-CEM cell and indicates that [¹⁸F]F—AraG uptake is dose dependent. These data also support that a majority of the [¹⁸F]F—AraG is taken up by cells within the first hour of exposure. The rapid uptake is necessary if [¹⁸F]F—AraG, with an isotope half life of 110 minutes, is eventually going to prove efficacious as a PET tracer. Having ascertained that lymphoblastic T cell lines will take up [¹⁸F]F—AraG, we then looked to see if primary T cells, non neoplastic T cells derived from normal mouse tissue, would also uptake [¹⁸F]F—AraG. FIG. 3.3 represents the data of two independent experiments indicating that non neoplastic but activated primary T cells will take up [¹⁸F]F—AraG to an appreciable level. The increased uptake of [¹⁸F]F—AraG by activated T cells may enable one to utilize [¹⁸F]F—AraG as a PET tracer in the detection of graft versus host disease (GVHD). GVHD is predominantly a T cell driven disease and the ability to detect aberrantly activated T cells by PET may facilitate an early diagnosis of GVHD in patients without invasive procedures. As AraG has been reported to induce neurotoxic side effects in some patients at therapeutic serum levels (~150 µM) [24], we chose to utilize doses lower (about 0.5 µM) than reported therapeutic levels of AraG in our assays to optimize [¹⁸F]F—AraG as a tracer for PET while avoiding the potential neurotoxicity in PET patients.

As discussed in this example, [¹⁸F]F—AraG can be synthesized by a direct fluorination method. This approach could be used for the synthesis of other important PET tracers such as [¹⁸F]FEAU, [¹⁸F]FMAU and [¹⁸F]FBAU (J. Label. Radiopharm. 2003, 46, 285-289, which is incorporated herein by reference) which are currently synthesized by multiple steps and involve lengthy purification processes. Preliminary cell uptake experiments done in CCRF-CEM cells (unactivated) and activated primary T Cells suggest application of the [¹⁸F]F—AraG as a new PET imaging agent for detection of disease of T cell origin.

### Example 3: Use of 18f-f-arag in Cardiac Imaging

FIG. 5 shows the kinetic properties of dCK and dGK for [³H]F—AraG. (a) Enzyme kinetics of dGK or dCK for [³H]F—AraG (b) dGuo positive control for dGK activity (c) dCyd positive control for dCK activity. Lines indicate best fit. The Km of dGK for [[³H]F—AraG was found to be 7.27 µM with a Vmax at 23.44 nmol/min/mg, while dCK had lower affinity for [³H]F—AraG (Km═50.89 µM, Vmax= 262.5). We observed high affinity of dCK for dCyd (Km=1.997, Vmax= 9.454) and similarly higher affinity in the dGuo positive control for dGK activity (Km═ 5.083, Vmax= 123.2).

Because dGK has a higher affinity for [¹⁸F]F—AraG, we considered the possibility that, at tracer levels, [¹⁸F]F—AraG may preferentially be phosphorylated by the mitochondrial kinase. Mitochondria are organelles that are responsible for supplying cells with energy by generating ATP. Heart, with its high energy needs, is an organ particularly rich in mitochondria. In the past decade, mitochondrial dysfunction has been recognized as an important aspect of cardiovascular pathology. Consequently, treatments focusing on improving heart’s mitochondrial function are rapidly emerging.

As a substrate for mitochondrial dGK, [¹⁸F]F—AraG has been discovered to be an agent useful to report on mitochondrial activity in the heart.

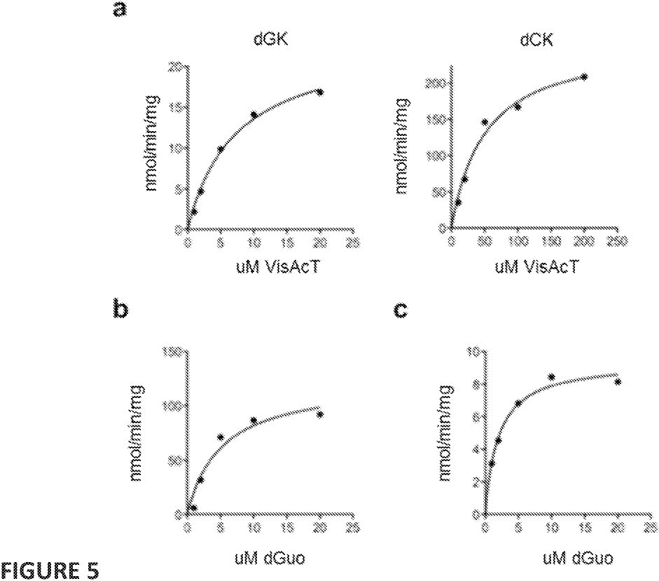

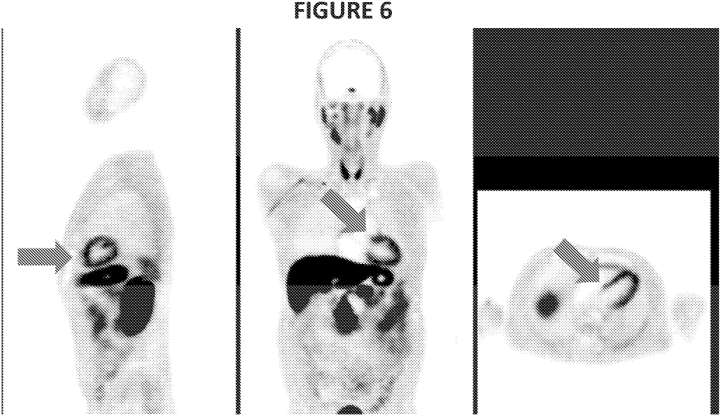

In an illustrative embodiment of the invention, the use of [¹⁸F]F—AraG as a tracer in PET methodologies was evaluated in healthy volunteers and cancer patients undergoing immunotherapy. Prominent uptake in the heart was observed in both groups (FIG. 6). FIG. 6 shows A PET method using [¹⁸F]F—AraG as a tracer in the heart. Our disclosure provides evidence that the high mitochondrial activity of the heart apparently results in high [¹⁸F]F—AraG signal one hour post injection of the tracer.

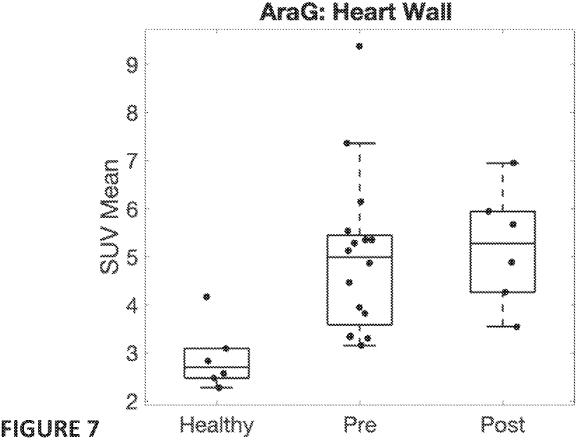

Interestingly, in this embodiment of the invention the [¹⁸F]F—AraG signal in the heart wall of healthy volunteers was discovered to be different than in patients undergoing immunotherapy. In particular, FIG. 7 shows the intensity of the [¹⁸F]F—AraG signal in the heart wall of healthy volunteers and patients undergoing immunotherapy (pre refers to a pre-treatment image and post to an image taken 2-3 weeks after one injection of anti PD-1 antibody).

The results provided herein show [¹⁸F]F—AraG′s ability to image heart and report on the changes that might be caused by drugs or injury. In view of this, using [¹⁸F]F—AraG as a PET tracer can offer significant advantages over the existing ¹⁸F labeled tracers in several distinct applications including myocardial perfusion, myocardial viability and heart inflammation, as well as cardiac drug development studies and methods to observe the treatment response to mitochondria targeting drugs.

### Example 4: [18f]f-arag As an Imaging Biomarker For Early Diagnosis And Monitoring of Cardiotoxicity Related to Doxorubicin and Immune Check Point Inhibitor Therapy

This example (certain aspects of which are prophetic) discloses the development of a sensitive and specific PET imaging strategy for early diagnosis of cancer therapy-associated cardiac toxicity. We focus on two classes of cancer therapies associated with cardiotoxicity: such as administration of immune checkpoint inhibitors (ICI), which lead to myocardial injury via infiltration of T cells resulting in myocarditis, as well as anthracyclines (e.g. doxorubicin) which cause mitochondrial dysfunction and direct myocyte death. In this example we discuss: 1) how [¹⁸F]F—AraG can allow early and pathophysiology-specific identification of cardiac toxicity related to both ICI and doxorubicin treatment; 2) Comparisons of ¹⁸FDG and [¹⁸F]F—AraG and determine variability in [¹⁸F]F—AraG myocardial uptake; and 3) determining the range of normal physiological [¹⁸F]F—AraG uptake in healthy subjects in order to, for example, perform retrospective analysis of myocardial uptake in cancer patients.

Life expectancy after cancer diagnosis has markedly improved through earlier diagnosis and more effective cancer therapies. However, general toxicity of cancer treatments can interfere with therapy, affect survival and lead to debilitating adverse effects and diminished quality of life. Therapy associated cardiovascular toxicities are a major cause of morbidity and mortality in cancer patients and cancer survivors (1). For many cancer survivors, cardiovascular events, and not cancer recurrence, represent the major risk of death in many types of cancer (2). Cardiovascular complications can arise with traditional cancer therapies, such as anthracyclines, as well as more selective, targeted therapies, such as kinase inhibitors, have been well documented (3,4). Immune checkpoint inhibitors (ICI), monoclonal antibodies that target regulators of the immune response, such as programmed death-1 (PD-1), its ligand (PD-L1), or cytotoxic T lymphocyte antigen-4 (CTLA-4), have led to impressive clinical outcomes in a fraction of patients with advanced tumors, but multisystem immune-related adverse effects, including cardiovascular toxicity, have been reported with treatment (5). The ICI-related cardiovascular toxicities have not been well delineated and are considered to be largely under-reported. Of all immune-related adverse effects, myocarditis is the most lethal, with a fatality rate close to 50% (6, 7). Considering rapidly expanding application of ICI in the treatment of cancer, the accurate assessment and better understanding of immune-related cardiac toxicity represents an unaddressed clinical challenge, crucial for successful patient care and management.

Due to the advantage of being noninvasive, cardiac imaging is the most recommended technique for monitoring cardiac function during and after cancer therapy (8,9). Serial measurement of left ventricular ejection fraction (LVEF) by echocardiography is currently the standard technique for detection of cardiotoxicity. However, reduction in LVEF is often a late manifestation of cardiac injury and may indicate irreversible damage. Cardiac magnetic resonance imaging allows detection of mechanical changes that occur before left ventricular dysfunction but lacks molecular specificity and sensitivity in ICI-related cardiotoxicity (10).

The inability of currently used imaging methods to detect subclinical cardiac involvement represents a major bottleneck for prevention and better management of cardiac complications in cancer patients and survivors (11). Moreover, with the advent of immunotherapies, prompt diagnosis of inflammatory cardiac sequelae is essential with current diagnostic techniques not being sensitive or specific. High specificity, resolution, and sensitivity make positron emission tomography (PET) the gold standard imaging modality for assessment of myocardial metabolism and perfusion, but evaluation of its utility in early detection of cancer therapy related cardiac toxicity has been limited. Nonetheless, PET imaging agents that target early indicators of cardiovascular toxicity offer a powerful and highly specific noninvasive tool for detection of subclinical cardiac toxicity.

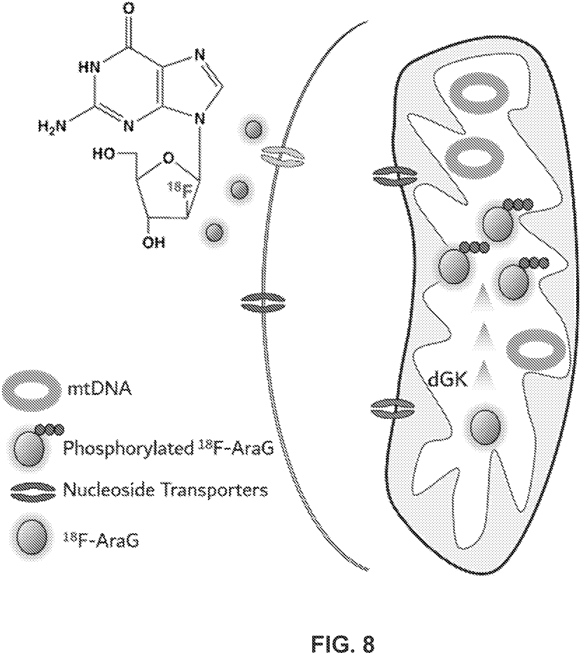

In this example, we focus on [¹⁸F]F—AraG, a PET agent with a unique ability to evaluate mitochondrial function in activated T cells and cardiomyocytes, as an imaging biomarker for early diagnosis and monitoring of cardiotoxicity associated with ICI as well as chemotherapy. [¹⁸F]F—AraG was developed by Namavari et al. for imaging activated T cells (12). It is a ¹⁸F-labeled analog of arabinosylguanasine (AraG), a compound that has shown remarkably selective accumulation in T cells (13,14) and whose prodrug, nelarabine, is used for treatment of T cell acute lymphoblastic leukemia and T cell lymphoblastic lymphoma. [¹⁸F]F—AraG enters T cells via nucleoside transporters and is trapped intracellularly through phosphorylation primarily by deoxyguanosine kinase (dGK), an enzyme present solely in mitochondria, and critical in supplying nucleotides for mitochondrial DNA synthesis (FIG. 8) (15-17). In response to activation, T cells undergo metabolic reprogramming and dramatically increase both mitochondrial mass and mtDNA (18,19) which can be visualized by [¹⁸F]F—AraG. The ability of [¹⁸F]F—AraG to image T cells activation and thus provide early indication of adaptive response to immunotherapies in cancer patients is currently being investigated in multiple Phase II trials.

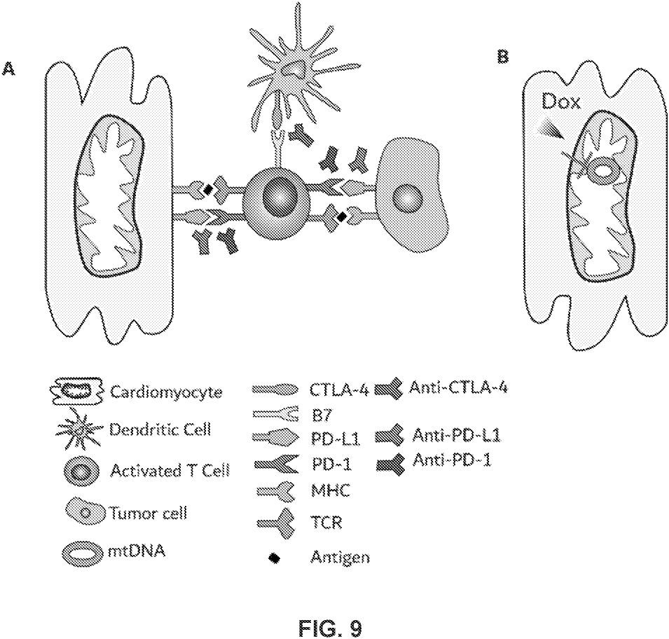

Myocarditis, the most common and fatal manifestation of ICI-associated cardiovascular toxicity, is thought to be a result of exaggerated adaptive response against the antigens shared between myocardium and tumor cells (FIG. 9A) (20). Infiltrating T cells have been implicated in the pathogenesis of heart failure (21), and have been detected in cases of fatal ICI-myocarditis (7, 22). Without being bound by a specfic theory or mechanism of action, we believe that [¹⁸F]F—AraG is able to simultaneously detect infiltration of activated T cells in tumors and the heart and thus serve as a tool for early detection of ICI-related cardiotoxicity.

As a substrate for mitochondrial dGK, an enzyme in the nucleotide salvage pathway responsible for supplying precursors for mtDNA synthesis in a rate limiting manner (23), [¹⁸F]F—AraG is uniquely suited to report on mitochondrial status not only in activated T cells but also in cells high in mitochondria, such as the cardiomyocytes. Cardiotoxicity of doxorubicin, a widely used chemotherapeutic, has been thoroughly studied and although there is a plethora of proposed mechanisms, they all converge on dysregulation of mitochondrial function in cardiomyocytes (24). Doxorubicin interacts with mitochondria in multiple ways, affecting membrane potential, and leading to oxidation and depletion of mitochondrial DNA (mtDNA) (FIG. 9B) (25). Our disclosure provides evidence that the aberrant mitochondrial function in doxorubicin-associated cardiomyopathy can be visualized by [¹⁸F]F—AraG, enabling early detection of doxorubicin-cardiotoxicity.

The ability to evaluate cardiac toxicity associated with both ICI and doxorubicin is highly significant in the context of increasing use of chemo/immunotherapy combinatorial approaches (26) that lead to potentiated damage to the heart, caused by cardiomyocyte injury by chemotherapy as well as by effector T cells.

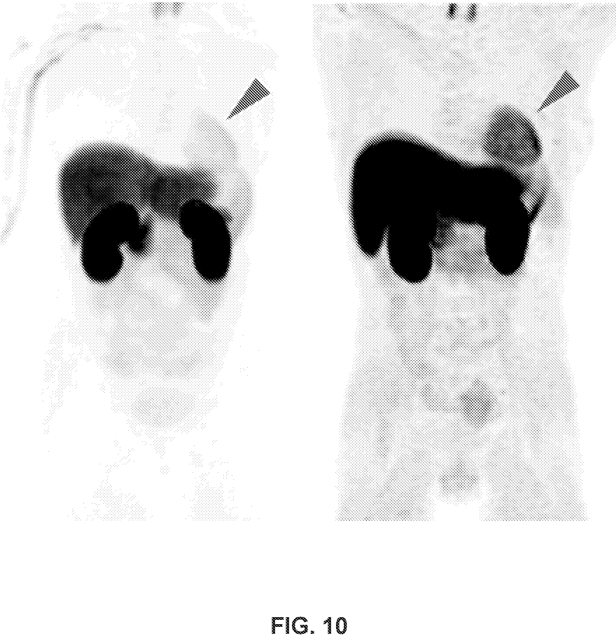

The capacity of [¹⁸F]F—AraG to asses mitochondrial status of cardiomyocytes has been indicated by the myocardial uptake observed in healthy human volunteers. Remarkably, compared to the healthy subjects, some patients undergoing immunotherapy showed significantly higher signal in the heart wall, indicating the ability of [¹⁸F]F—AraG to detect aberrant mitochondrial and/or inflammatory activity (FIG. 10).

In this example, we consider the underlying mechanism of the increased [¹⁸F]F—AraG uptake to better understand the observed clinical findings. The ability to accurately interpret myocardial uptake detected in patients undergoing immunotherapy is of high significance as it can provide an assessment of a clinic-ready tool to simultaneously report not only on therapy response but on adverse effects of therapy as well. [¹⁸F]F—AraG can fill an urgent clinical need for noninvasive measurement of mitochondrial function, that can also aid in development and more successful translation of mitochondria-targeted therapies to humans (27). The capability of [¹⁸F]F-AraG PET to detect early cardiac involvement can have an immense impact on patient management, with a potential to revolutionize cardio-oncology, a rapidly advancing field focused on balancing therapeutic efficacy and cardiovascular safety. [¹⁸F]F-AraG PET can allow optimization of therapy, reduction of cardiovascular risk and cardiovascular disease-related morbidity and mortality, and improvement in quality of life in cancer patients and survivors. These benefits to patients can in turn reduce the high healthcare and societal costs associated with cardiovascular disease.

Currently used imaging methods lack molecular specificity to detect subclinical cardiac involvement associated with cancer therapy. The exceptional advantage that this offers is apparent from preliminary clinical data, data that indicate great potential of [¹⁸F]F-AraG PET to detect irregular mitochondrial activity in the heart. In this context, [¹⁸F]F—AraG may currently be the only agent in the clinic, able to report on the damage caused by a variety of cancer treatments: chemo-, targeted and ICI. In addition, an ability to simultaneously report on immune response in target tissue and off-target toxic effects of immune therapy is a characteristic unique to [¹⁸F]F—AraG. This example offers development of a new method for assessment of cardiotoxicity associated with ICI- and combinatorial chemo/ICI approaches for which no diagnostic approaches currently exist.

### Embodiment 1. Demonstrate That [¹⁸F]F-AraG Allows Early Identification of Cardiac Toxicity Related to Pathophysiology of Mitochondria Injuring Drugs (e.g ICI and Doxorubicin)

[¹⁸F]F—AraG imaging can correlate with biomarkers of cardiac pathophysiology and enable early detection of cardiotoxicity.

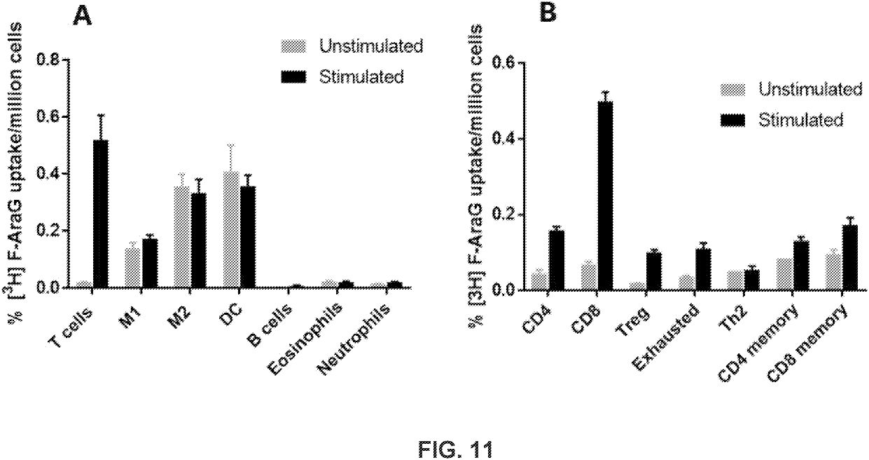

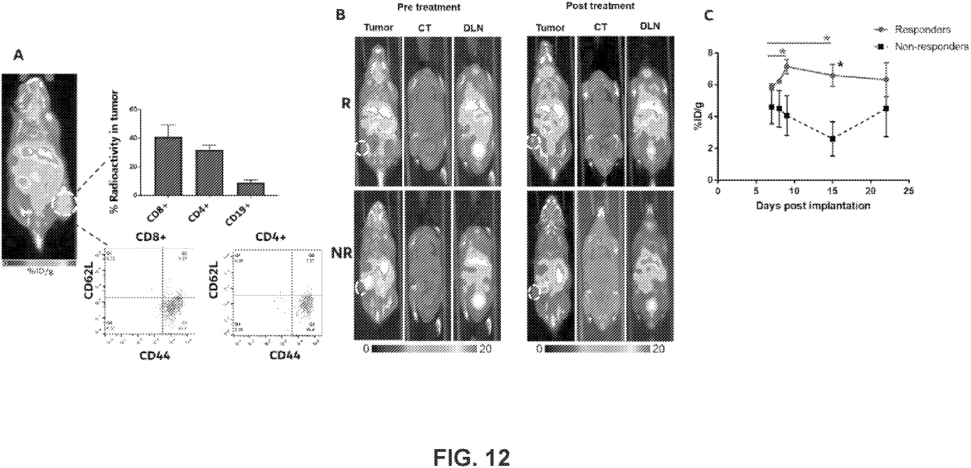

### Rationale and Supporting Evidence.

[¹⁸F]F—AraG has been comprehensively evaluated in cell culture and in preclinical immune cell-mediated disease models - graft versus host disease and rheumatoid arthritis (28,29). [¹⁸F]F—AraG preferentially accumulates in activated CD8+ cells, although some accumulation is also observed in macrophages and dendritic cells (FIG. 11). As T cells play a vital role in immune response to tumors, we focused on determining the value of [¹⁸F]F—AraG in immuno-oncology (30,31). Preclinical studies confirmed [¹⁸F]F-AraG’s specificity for activated T cells and utility in predicting response to immunotherapy (30). Using rhabdomyocarcoma model, we determined that more than 80% of the detected intratumoral signal came from the tracer accumulation in immune cells, primarily activated CD8+ and CD4+ (FIG. 12). Importantly, [¹⁸F]F—AraG signal detected in the colon cancer tumors and in the tumor draining lymph nodes was able to distinguish between responders and non-responders to anti-PD-1 therapy early in the treatment, only 48 hours after single therapy administration (FIG. 12).

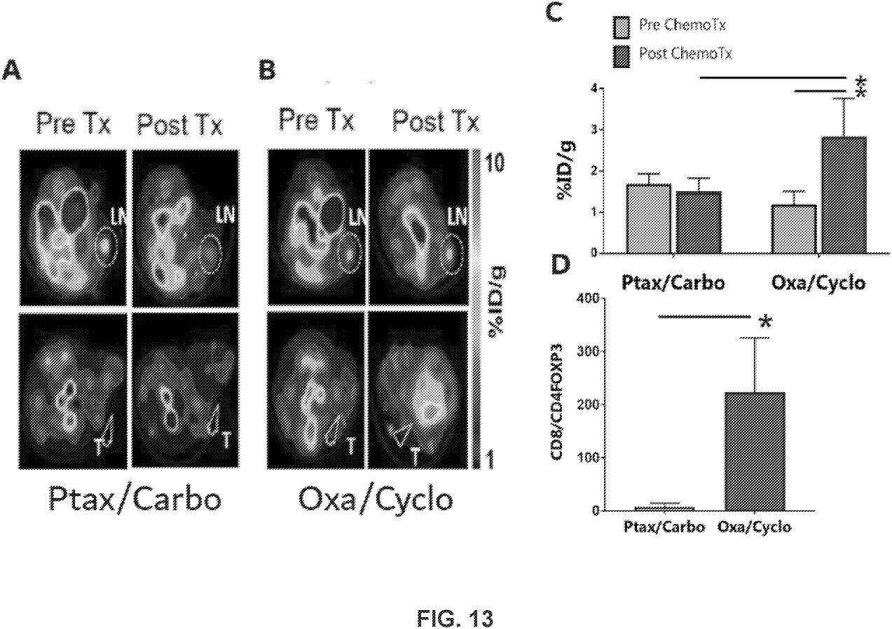

The discovery of the immune response that results from chemotherapy-induced tumor cell death, termed immunogenic cell death (32), has sparked interest in harnessing chemotherapy’s immunomodulatory effects for possible synergistic combinations with immunotherapeutics (33,34). Longitudinal [¹⁸F]F—AraG imaging of colon cancer model undergoing two types of chemotherapy, one known to induce immunogenic cell death and the other reported to cause immunogenically silent tumor death revealed dramatic differences in immunomodulatory effects caused by the two chemo regimens and demonstrated [¹⁸F]F—AraG′s utility to evaluate chemotherapy-induced immunomodulation and an immune active environment (FIG. 13) (31).

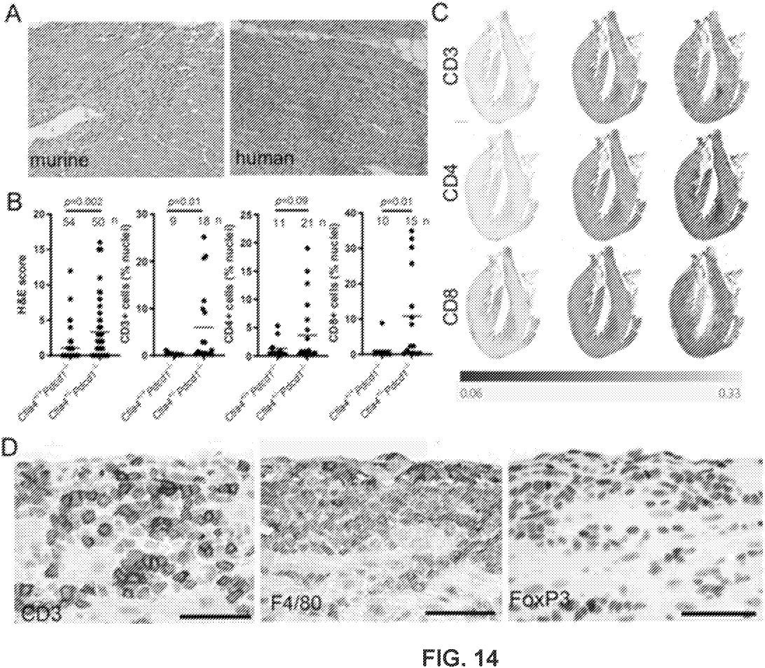

Checkpoints, such as PD-1 and CTLA-4 that play an immunosuppressive role in antitumor immunity, have also been shown to be critical players in peripheral immune tolerance to the heart and prevention of autoimmune myocarditis. ICI drugs that disrupt PD-1 and CTLA-4 signaling can lead to a breakdown of peripheral immune tolerance and excessive myocardial T cell infiltration and activation resulting in myocarditis. Artisans have created both pharmacological and genetic models of ICI-myocarditis by treatment of mice with anti-CTLA-4 and anti-PD-1 antibodies as well as genetically modifying the ICI targets. Notably, haploinsufficiency in the CTLA-4 gene (Ctla4+/-) combined with PD-1 deficiency (Pdcd1-/-) results in myocarditis in ~50% of mice which phenocopies ICI-myocarditis in patients (FIG. 14) (35). Strikingly, the autoimmunity in Ctla4+ /- Pdcd1-⁻ mice is restricted to the restricted to the cardiovascular system and characterized by T cell and myeloid infiltration into the myocardium, as observed in humans.

Although the exact mechanisms of doxorubicin-associated cardiac injury are still debated, many studies suggest mitochondria as the main target in doxorubicin-induced damage of cardiomyocytes (25,36,37). Doxorubicin readily enters mitochondria, and can form adducts with mtDNA and cardiolipin, inhibiting mitochondrial respiration. Mitochondrial dysfunction appears to be one of the earliest indicators of doxorubicin-associated cardiomyopathy (38). Preclinical studies with agents that evaluate cardiac mitochondrial membrane potential, ¹⁸F-Mitophos and ⁶⁸Ga-Galmydar, showed a reduction in cardiac uptake in doxorubicin treated animals, indicating a potential of mitochondrial imaging in chemotherapy-induced cardiotoxicity (38,39). Interestingly, doxorubicin treatment led to an increase in ¹⁸FDG uptake in the heart, suggesting changes in metabolism (40).

The demonstrated capability of [¹⁸F]F—AraG to detect activated T cells and evaluate mitochondrial function, both of which play a central role in mechanisms of cardiotoxicity (FIG. 9), indicate its potential to serve as a pathophysiology-related imaging biomarker for early detection and monitoring of cardiotoxicity associated with ICI and doxorubicin therapy. The ability to evaluate clinically relevant cardiotoxicity associated with both chemo- and immuno-therapeutics can be invaluable considering the lack of knowledge on cardiovascular damage of rapidly growing chemo/immunotherapy combinatorial approaches.

One embodiment of the invention can be used to determine the correlation between [¹⁸F]F—AraG′s myocardial uptake and pathophysiology of cardiac toxicity. To show that [¹⁸F]F—AraG can allow early and specific imaging of cardiotoxicity, we can use [¹⁸F]F—AraG to monitor the development of cardiotoxicity caused by ICI, doxorubicin, and doxorubicin/ICI treatment and compare imaging findings to the current standard-of-care measurement of LVEF, and correlate with known markers of cardiac damage.

### Experimental Approach

Animal Models of Cardiotoxicity. To investigate dose-dependent doxorubicin cardiotoxicity, mice (n=8 per group, 4 male, 4 female) can be treated with low (4 mg/kg) and high (20 mg/kg) dose of doxorubicin. Control mice (n=8, 4 male, 4 female) can receive only vehicle injections. For the study of ICI-related myocarditis, we can use a recently developed preclinical model (35). Ctla4+/- Pdcd1-/- mice fully recapitulate the hallmark pathophysiological and clinical characteristics of ICI-myocarditis. Ctla4^{+/+}Pdcd1⁻ ^{/-}mice can be used as controls. We can use 8 Ctla4 +/- Pded1-/- mice (4 male, 4 female) and 8 Ctla4^{+/+}Pdcd1^{-/-} control mice.

In addition, artisans have generated a milder form of ICI-myocarditis by treatment of mice in specific genetic backgrounds with anti-CTLA-4 and anti-PD-1 antibodies. MRL-Fas^{lpr} mice treated with anti-CTLA-4 or anti-PD-1 monotherapy, or combination therapy twice weekly for 8 weeks did not display obvious clinical signs but histological and electron microscopic examination revealed immune infiltration of the myocardium and vasculature, as well as signs of endothelial cell damage and sarcomere disarray following combination therapy. We believe the combination of pharmacologic and genetic mice for modeling ICI-myocarditis are complementary. Finally, as the use of ICI expands to cancer types where patients may be exposed to a combination of doxorubicin and ICI (e.g., triple negative breast cancer), these mice provide a platform where the cardiovascular effects of combination therapies can be explored. To investigate cardiotoxicity of the combinatorial doxorubicin/ICI treatment we can use 16 MRL-Fas^{lpr} mice. Eight mice (half male, half female) can be treated with doxorubicin (20 mg/kg), and anti-PD-1 and anti CTLA-4 antibodies (250 µg). MRL-Fas^{lpr} mice treated with vehicle can serve as a control.

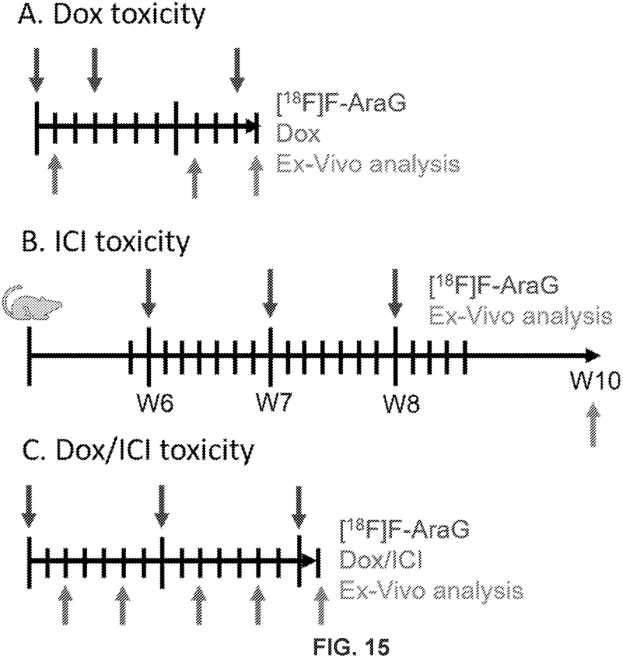

PET and LVEF Imagine. [¹⁸F]F—AraG can be produced by conventional methods for example at the UCSF Radiochemistry Core. For PET/CT, mice can be injected with ~200 uCi (7.4 MBq) in ~0.1 ml of [¹⁸F]F—AraG via tail vein and imaged using a dedicated small animal PET/CT (Siemens Inveon). In a subset of mice, we can perform 60-minute dynamic PET imaging followed by CT at the baseline and two post therapy time points. Electrocardiogram-gated PET can be performed after the last imaging time point to capture the information of left-ventricular ejection fraction (LVEF). Timeline for longitudinal monitoring of cardiotoxicity for all three animal models is shown in FIG. 15. Briefly, pharmacologic models (doxorubicin and doxorubicin/ICI) can be imaged before and periodically during treatment. Genetic ICI myocarditis model can be imaged three times once a week, starting in week 6. The list mode PET data of 60 minutes can be reconstructed to dynamic multiframes (12×5, 6×10, 4×30, 6×60, and 10×300 s) using the 3D ordered subsets expectation maximization with maximum a posteriori (OSEM/MAP) algorithm provided by the scanner manufacturer. Attenuation and scatter corrections to ensure quantitative accuracy can be applied using CT-based attenuation map and scatter model. ECG-gated data with 8 cardiac bins can be reconstructed using the same algorithm and corrections. Dynamic multiframe data can be used for two-tissue compartment model, and the last 20 minutes of the data can be used for static data analysis. Rate constants including influx rate constant (Ki) can be derived from dynamic data, and %Injected Dose/g (%ID/g) can be derived from static data. LVEFs can be calculated from the ECG-gated data. All of these parameters can be mapped to polar plots for regional and segmental analysis of the parameters. Image processing can be performed on PMOD/PCARD/PKIN (PMOD Technologies).

### Embodiment 2. Compare ¹⁸F-fluorodeoxyglucose (¹⁸FDG) and [¹⁸F]F—AraG and Determine Variability in [¹⁸F]F-AraG Myocardial Accumulation

[¹⁸F]F—AraG can allow blood glucose-independent evaluation of cardiac function and show low signal variability.

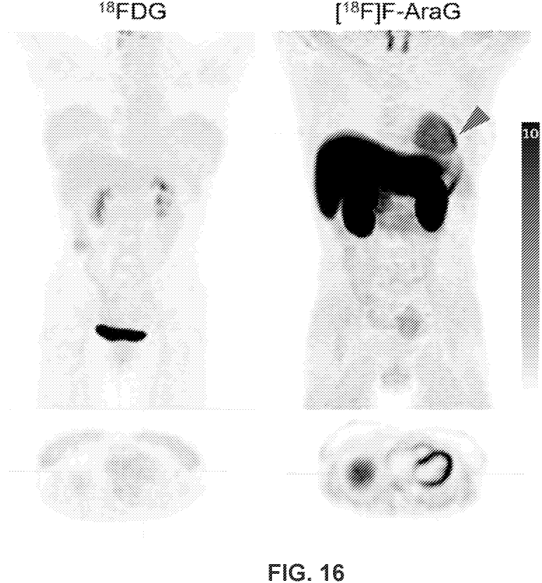

Rationale and Preliminary Evidence. ¹⁸FDG PET is routinely used in diagnosis, staging and restaging of oncologic patients. The wide use of FDG led to incidental myocardial findings that prompted many studies aiming to assess the utility of FDG in cardiac imaging. ¹⁸FDG is now the most utilized and validated PET tracer for evaluation of myocardial metabolism and viability. A radiolabeled analog of glucose, ¹⁸FDG reports on utilization of glucose in tissues. To minimize interference from the blood glucose and achieve high sensitivity of detection in tumors that use glucose as a primary source of energy, ¹⁸FDG scan in oncologic patients is performed under fasting conditions. In the fasting state, cardiac ¹⁸FDG uptake is low, reflecting reduced glucose metabolism caused by a decrease in insulin and increase in fatty acids, heart’s major energy substrates (FIG. 16). Because myocardial FDG uptake depends on plasma glucose, fatty acid and insulin levels, but also on diet, activity and use of certain drugs, wide spatial and temporal variability in signal has been reported (42,43). The length of fasting investigated as a variable that can be controlled to afford a more specific and accurate distinction between benign and malignant myocardial uptake does not appear to correlate with myocardial glucose metabolism (43). The good quality of ¹⁸FDG images is especially difficult to achieve in diabetic patients, a population with high rates of cardiovascular co-morbidity.

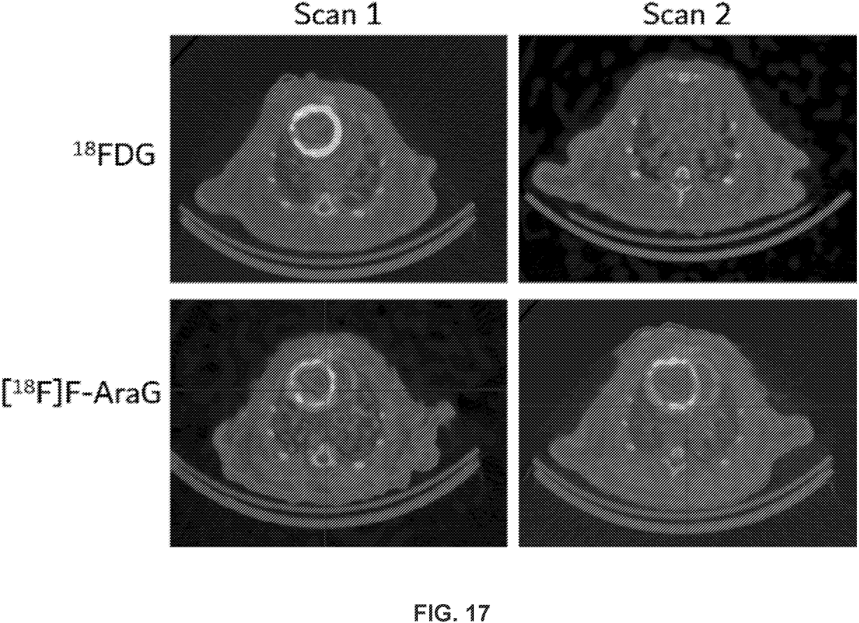

The mechanism of [¹⁸F]F—AraG uptake differs from the one of glucose (FIG. 8) and consequently signal variability, as it relates to fasting, is expected to be lower (FIG. 17). As a radiolabeled analog of a nucleoside - deoxyarabinoguansine, [¹⁸F]F-AraG’s uptake reflects the activity of the enzymes of the salvage pathway, considered to be the predominant process by which the purine nucleotides pools in the myocardium and lymphoid organs are maintained (44,45). Dietary nucleotides are the primary source of nucleosides that may compete with [¹⁸F]F—AraG in the salvage pathway and result in a diet-related signal variation.

One embodiment of the invention can determine the differences in signal variability between ¹⁸FDG and [¹⁸F]F—AraG and to better understand the potential sources of [¹⁸F]F—AraG signal heterogeneity. Aspects of the invention can be tested by determining the differences in ¹⁸FDG and [¹⁸F]F—AraG myocardial signal in animals exposed to different conditions - fasted and non-fasted, anesthetized and active, fed diet with and without dietary nucleotides.

### Experimental Approach

We can investigate the effect of three variables on the myocardial uptake: fasting, activity and diet. For each variable we can use 16 mice, 8 male and 8 female, that can be split into two groups. For the study of fasting one group can be allowed free access to both food and water and the other group can be fasted for 8 hours with access to water. For the study of activity, one group of animals can be kept under anesthesia and the other group can be allowed to wake up after the tracer injection and prior to imaging. To investigate the effect of dietary nucleotides, both groups of animals can be fed nucleotide free diet and only one supplemented with 0.04% nucleotides, a level found to be optimal for immune function (46).

PET Imaging. All animals can be imaged with ¹⁸FDG and [¹⁸F]F—AraG on two consecutive days. To asses temporal variability, imaging can be repeated 3 days after the initial scan. PET data can be acquired for 10 minutes static, and reconstructed using the 3D OSEM/MAP. Attenuation and scatter corrections to ensure quantitative accuracy can be applied using CT-based attenuation map and scatter model. %ID/g can be derived from static data. The variability in signal intensity and location can be determined for each tracer and values compared. The effect of the variables on the signal intensity can be determined for each tracer. Uptake values can be calculated for the heart, muscle, liver, spleen, kidney and brain.

Blood glucose and nucleotide measurements. The glucose levels can be determined in the tail vein blood sample immediately before imaging using glucose meter. Measurement of nucleotides in the blood requires large volume of analyte and can thus be measured after the final PET scan, following terminal intracardiac blood collection (47).

Image quantification and sample size calculation. %ID/g between the group with each variable and the control group can be derived from the PET data. Our hypothesis is that we can not see a significance in [¹⁸F]F—AraG uptakes by the dietary variable. With the sample size of 8 per cohort, for significance level α = 0.05 and power (1-β) = 0.85, a two samples t-test can be statistically significant if the difference is over 30 % with 20% of standard deviation. Hence, if the difference is smaller than that, there is no statistical significance with power of 0.85, which is what we anticipate for this comparison study.

We expect to demonstrate: 1) no/minimal difference in [¹⁸F]F—AraG signal between fasted and non-fasted animals 2) no/minimal difference in [¹⁸F]F—AraG signal between anesthetized and active animals 3) the extent of the effect of dietary nucleotides on the [¹⁸F]F—AraG signal 4) lower variability in [¹⁸F]F—AraG signal compared to ¹⁸FDG. Overall, this can allow assessment of the advantages that [¹⁸F]F—AraG can offer over ¹⁸FDG as well as indicate a potential source of variation in [¹⁸F]F—AraG signal that does not relate to pathophysiology.

### Embodiment 3. Determine the Range of Physiological [¹⁸F]F-AraG Uptake in Healthy Subjects and Feasibility of Using Increased [¹⁸F]F-AraG Uptake as an Indication of Early Cardiotoxicity in Cancer Patients

The physiological myocardial ¹⁸F-FAraG uptake can be significantly lower than the uptake in cancer patients allowing assessment of cardiotoxicity. [18F]F—AraG uptake post treatment in cancer patients can be used for early assessment of cardiotoxicity (e.g. ICI or other drugs).

Rationale and Preliminary Evidence. For [¹⁸F]F—AraG to serve as a tool that can indicate cardiotoxicity with high specificity, several conditions need to be met: a) physiologic uptake in the normal heart should be relatively low and well defined, b) signal in the diseased state must differ significantly from the normal heart threshold c) signal characteristics (intensity, pattern) should correspond to pathophysiology. One embodiment of the invention can be used to better understand differences between physiologic and pathologic uptake in human subjects by assessing signal variability in healthy volunteers and performing retrospective analysis of cardiac accumulation in cancer patients.

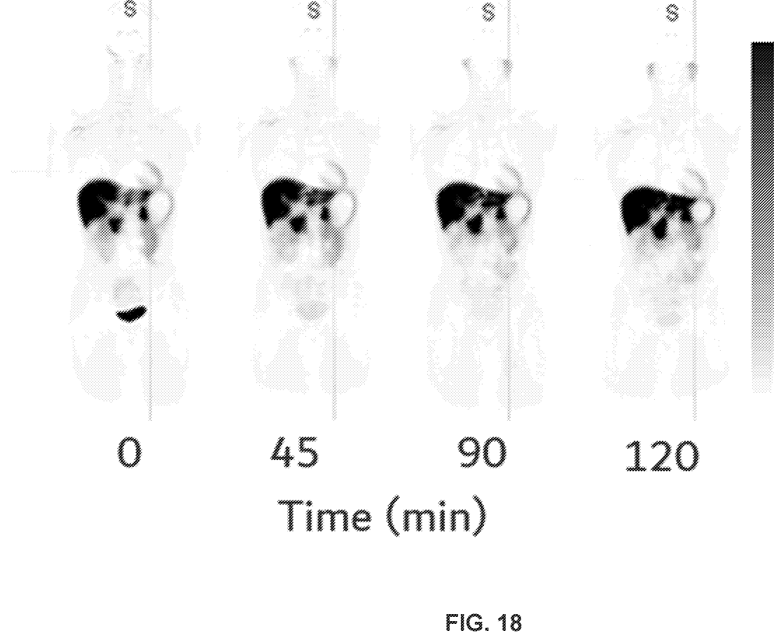

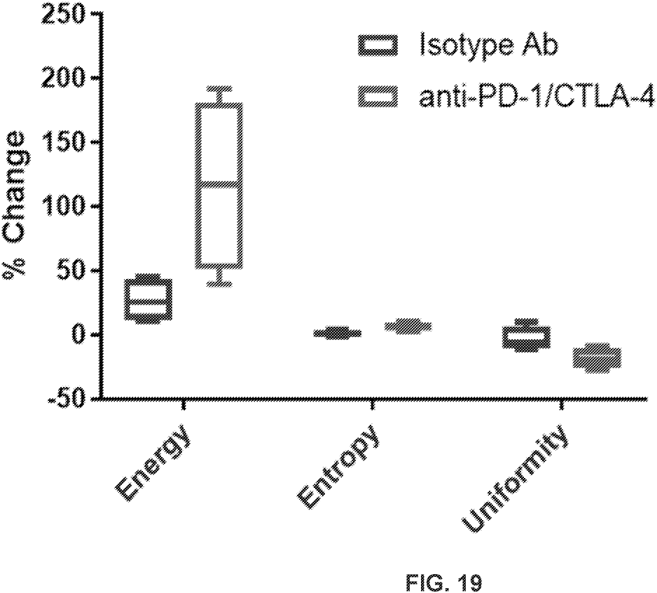

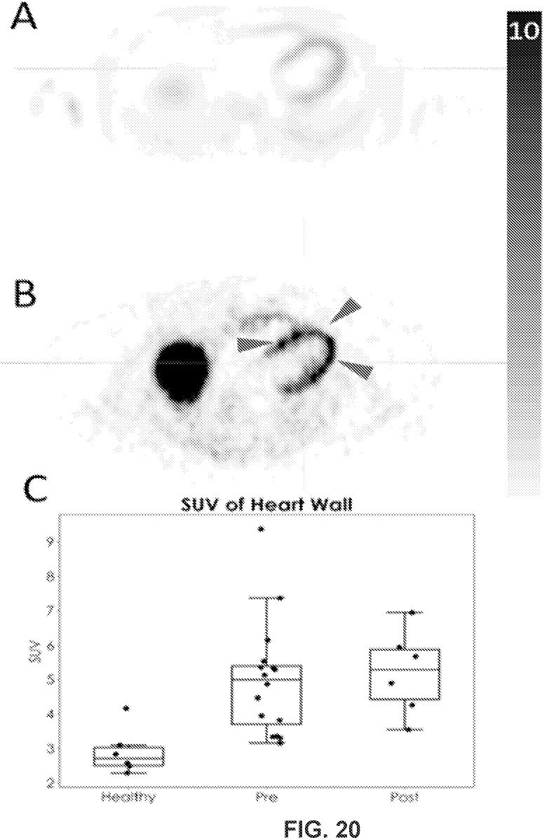

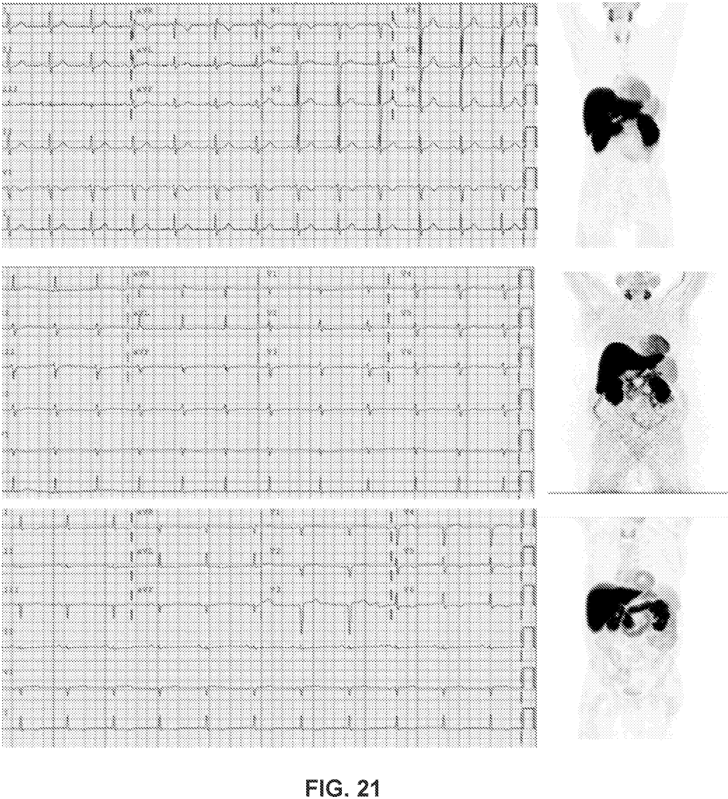

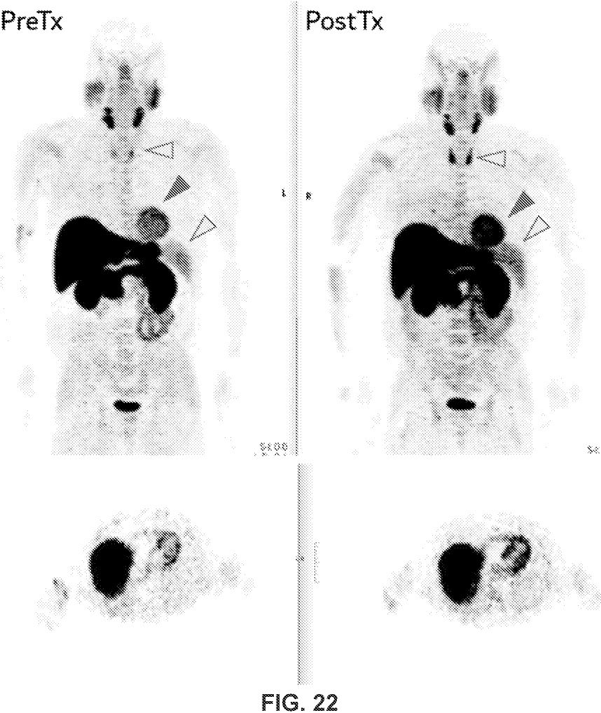

The safety and radiation dosimetry of [¹⁸F]F—AraG was studied in six, healthy subjects (three male and three female) during the first-in-human study at UCSF. The myocardial uptake was observed in all six subjects, with the mean SUV in the heart wall of 2.9 ± 0.62. The signal persisted during the imaging window, indicating trapping of the tracer (FIG. 18). The tracer uptake appeared homogenous with no focal or regional increases. Although the relatively low uptake and uniform signal in healthy subjects indicate a potential for detection of abnormal signal intensity and pattern in diseased states, the relatively small sample size may not be representative of the physiologic accumulation in a larger population. To more comprehensively evaluate baseline values, we can, for example, image additional 30 healthy subjects, 15 male, 15 female, and to analyze cardiac signal using traditional and radiomic methods. Radiomics, a computational method used to analyze and extract abundant quantitative features present in the images, such as texture, heterogeneity, and shape, has been mostly employed in oncologic imaging for improvement of cancer screening and early detection (48,49). Radiomics has also been used to identify immune contexture characteristics within the tumor microenvironment for more accurate assessment of immunotherapy response (50,51). To date, the application of radiomics in cardiac imaging has been primarily focused on the analysis of CT and MRI images without the use of molecular biomarkers. Here, we disclose radiomic analysis of [¹⁸F]F-AraG PET images to extract image characteristics with strong biological relevance, that may provide a more accurate and pathology-specific assessment of cardiotoxicity. In our preliminary preclinical study, mice treated with anti-PD-1/CTLA-4 immunotherapy showed significant differences in intratumoral signal energy, entropy and uniformity from untreated mice (FIG. 19). Texture analysis of images can allow better characterization and quantification of the signal differences observed between healthy volunteers and some cancer patients (FIG. 20). [¹⁸F]F—AraG is currently being tested in multiple Phase II clinical trials that evaluate the utility of [¹⁸F]F—AraG in assessing response to immunotherapy in cancer patients. To date, over 40 patients with different types of cancer (squamous cell carcinoma of the head and neck, lung, bladder, breast, melanoma) have safely been imaged with [¹⁸F]F—AraG. We can perform a comprehensive retrospective analysis of myocardial uptake in [¹⁸F]F—AraG scans collected to date to allow comparative examination of the findings with a healthy cohort. Our preliminary analysis showed a significantly higher myocardial signal in cancer patients than in healthy subjects, both pre-and post-one infusion of immunotherapy (FIG. 20). The higher signal in pre-immunotherapy scans may be the indication of cardiotoxicity of a prior anticancer treatment, such as chemotherapy or radiotherapy. Within the limited patient cohort with accompanying electrocardiogram (ECG), heterogenous and more intense myocardial uptake seemed to correlate with abnormal ECG findings (FIG. 21). Importantly, in some patients, only one infusion of immunotherapy, resulted in a dramatically increased myocardial uptake (FIG. 22). Overall, these results illustrate the potential of [¹⁸F]F—AraG as a tool for cardiovascular risk stratification and assessment of early cardiotoxicity.

### Experimental Approach

Clinical Trial Outline. Study participants can undergo whole body PET/CT scans with [¹⁸F]F—AraG at the UCSF Nuclear Medicine Clinic. The study population can consist of 30 healthy subjects, 15 male and 15 female, with no known heart disease. To account for differences that may be related to age, within each gender group there can be three age groups: a) 18-40, b) 40-65 and c) over 65 years of age. Each age group can have 10 subjects,5 male and 5 female. Careful medical history can be obtained for each subject with special emphasis placed on familial history of heart disease and the use of medications and supplements. All cardiac medications and previous cancer therapies can be documented and collected on each patient. Subjects can be asked to fast for 6 hours to minimize potential variability coming from diet.

PET Imaging. [¹⁸F]F—AraG can be synthesized on a per patient, per scan basis prior to imaging. 5 ± 0.5 mCi of [¹⁸F]F—AraG can be administered as a bolus intravenous injection. Dynamic imaging can be performed in two female and two male patients from each age group (12 total). Dynamic data collection can start immediately after the tracer injection and can last for 60 minutes focusing on the heart field of view. Static images in 18 patients (3 male and 3 female from each age group) can be acquired 60 minutes post tracer injection. A non-contrast CT scan (5-mm contiguous axial cuts) (CTAC) can be obtained of the relevant region in helical mode using 120 kVp, 40 mAs, and a 512 × 512 matrix size, acquiring a field of view (FOV) of 38.5 mm/second. The CTAC images can be fused to the PET images and used for attenuation correction and anatomical localization. Immediately after the CTAC, an emission PET scan can be acquired in 3-dimensional Time of Flight (TOF) mode over the same anatomic regions. The PET emission scan can be attenuation corrected using segmented attenuation data of the CTAC scan.

Image Analysis. The PET images can be reconstructed with a standard iterative algorithm provided by the scanner manufacturer for both dynamic and static imaging data. From dynamic imaging, dynamic multiframes (12×5, 6×10, 4×30, 6×60, and 10×300 s) can be generated for tracer kinetic modeling (two-tissue compartment model). The last 10-min summed data can be also generated to represent comparable static data. Rate constants including influx rate constant (Ki) can be computed using PMOD/PCARD/PKIN (PMOD Technologies) in myocardium. The whole-body imaging from static scan is intended as how [¹⁸F]F—AraG is used for both tumor imaging and myocardial imaging. For this example, we can add additional reconstruction focusing on the same field of view of dynamic imaging, and the following standardized uptake value measures: SUVmax, SUVpeak, and SUVmean can be computed for the left ventricle. American Heart Association (AHA) 17-segment model can be used to quantify these values for static images. The kinetic parameters from dynamic imaging can be correlated with SUVs from static images to ensure if the static scan provides stable image quantification, not necessitating dynamic scans for practical implementation.

Radiomics analysis can be performed using the PyRadiomics library(52) as it is built on a non-proprietary software package (LifeX or 3D-slicer). Prior to extracting radiomics features, PET images can be normalized to SUV units and resampled to a common voxel size of 1 mm. Radiomics feature vectors can be computed for each volumetric lesion. A complete list of the radiomics features as defined in PyRadiomics can be extracted (53).

With embodiments of the invention, artisans can demonstrate: 1) relatively low and homogenous myocardial uptake in healthy subjects 2) narrow range in [¹⁸F]F-AraG myocardial uptake in healthy subjects 4) significantly higher cardiac uptake in cancer patients than in healthy population.

### References

1. Curigliano G, Lenihan D, Fradley M, et al. Management of cardiac disease in cancer patients throughout oncological treatment: ESMO consensus recommendations. Ann Oncol. 2020;31:171-190.

2. Carver JR, Shapiro CL, Ng A, et al. American Society of Clinical Oncology clinical evidence review on the ongoing care of adult cancer survivors: cardiac and pulmonary late effects. J Clin Oncol. 2007;25:3991-4008.

3. McGowan JV, Chung R, Maulik A, Piotrowska I, Walker JM, Yellon DM. Anthracycline Chemotherapy and Cardiotoxicity. Cardiovasc Drugs Ther. 2017;31:63-75.

4. Moslehi JJ. Cardiovascular Toxic Effects of Targeted Cancer Therapies. N Engl J Med. 2016;375:1457-1467.

5. Ramos-Casals M, Brahmer JR, Callahan MK, et al. Immune-related adverse events of checkpoint inhibitors. Nat Rev Dis Primers. 2020;6:38.

6. Wang DY, Salem JE, Cohen JV, et al. Fatal Toxic Effects Associated With Immune Checkpoint Inhibitors: A Systematic Review and Meta-analysis. JAMA Oncol. 2018;4:1721-1728.

7. Moslehi JJ, Brinkley DM, Meijers WC. Fulminant Myocarditis: Evolving Diagnosis, Evolving Biology, Evolving Prognosis. J Am Coll Cardiol. 2019;74:312-314.

8. Zamorano JL, Lancellotti P, Rodriguez Munoz D, et al. 2016 ESC Position Paper on cancer treatments and cardiovascular toxicity developed under the auspices of the ESC Committee for Practice Guidelines: The Task Force for cancer treatments and cardiovascular toxicity of the European Society of Cardiology (ESC). Eur J Heart Fail. 2017;19:9-42.

9. Armenian SH, Lacchetti C, Barac A, et al. Prevention and Monitoring of Cardiac Dysfunction in Survivors of Adult Cancers: American Society of Clinical Oncology Clinical Practice Guideline. J Clin Oncol. 2017;35:893-911.

10. Zhang L, Awadalla M, Mahmood SS, et al. Cardiovascular magnetic resonance in immune checkpoint inhibitor-associated myocarditis. Eur Heart J. 2020;41:1733-1743.

11. Steingart RM, Chandrashekhar Y, Marwick TH. Imaging in Cardio-Oncology: Where Are We and Where Should We Be Going? JACC Cardiovasc Imaging. 2018;11:1209-1211.

12. Namavari M, Chang YF, Kusler B, Yaghoubi S, Mitchell BS, Gambhir SS. Synthesis of 2′-deoxy-2′-[18F]fluoro-9-beta-D-arabinofuranosylguanine: a novel agent for imaging T-cell activation with PET. Mol Imaging Biol. 2011;13:812-818.

13. Lambe CU, Averett DR, Paff MT, Reardon JE, Wilson JG, Krenitsky TA. 2-Amino-6-methoxypurine arabinoside: an agent for T-cell malignancies. Cancer Res. 1995;55:3352-3356.

14. Eriksson S, Arner E, Spasokoukotskaja T, et al. Properties and levels of deoxynucleoside kinases in normal and tumor cells; implications for chemotherapy. Adv Enzyme Regul. 1994;34:13-25.

15. Shewach DS, Daddona PE, Ashcraft E, Mitchell BS. Metabolism and selective cytotoxicity of 9-beta-D-arabinofuranosylguanine in human lymphoblasts. Cancer Res. 1985;45:1008-1014.

16. Wang L. Mitochondrial purine and pyrimidine metabolism and beyond. Nucleosides Nucleotides Nucleic Acids. 2016;35:578-594.

17. Kim W, Le TM, Wei L, et al. [18F]CFA as a clinically translatable probe for PET imaging of deoxycytidine kinase activity. Proc Natl Acad Sci U S A. 2016;113:4027-4032.

18. Tan H, Yang K, Li Y, et al. Integrative Proteomics and Phosphoproteomics Profiling Reveals Dynamic Signaling Networks and Bioenergetics Pathways Underlying T Cell Activation. Immunity. 2017;46:488-503.

19. Baixauli F, Acin-Perez R, Villarroya-Beltri C, et al. Mitochondrial Respiration Controls Lysosomal Function during Inflammatory T Cell Responses. Cell Metab. 2015;22:485-498.

20. Khunger A, Battel L, Wadhawan A, More A, Kapoor A, Agrawal N. New Insights into Mechanisms of Immune Checkpoint Inhibitor-Induced Cardiovascular Toxicity. Curr Oncol Rep. 2020;22:65.

21. Blanton RM, Carrillo-Salinas FJ, Alcaide P. T-cell recruitment to the heart: friendly guests or unwelcome visitors? Am J Physiol Heart Circ Physiol. 2019;317:H124-H140.

22. Johnson DB, Balko JM, Compton ML, et al. Fulminant Myocarditis with Combination Immune Checkpoint Blockade. N Engl J Med. 2016;375:1749-1755.

23. Leanza L, Ferraro P, Reichard P, Bianchi V. Metabolic interrelations within guanine deoxynucleotide pools for mitochondrial and nuclear DNA maintenance. J Biol Chem. 2008;283:16437-16445.

24. Varga ZV, Ferdinandy P, Liaudet L, Pacher P. Drug-induced mitochondrial dysfunction and cardiotoxicity. Am J Physiol Heart Circ Physiol. 2015;309:H1453-1467.

25. Wallace KB, Sardao VA, Oliveira PJ. Mitochondrial Determinants of Doxorubicin-Induced Cardiomyopathy. Circ Res. 2020;126:926-941.

26. Xin Yu J, Hodge JP, Oliva C, Neftelinov ST, Hubbard-Lucey VM, Tang J. Trends in clinical development for PD-⅟PD-L1 inhibitors. Nat Rev Drug Discov. 2020;19:163-164.

27. Tian R, Colucci WS, Arany Z, et al. Unlocking the Secrets of Mitochondria in the Cardiovascular System: Path to a Cure in Heart Failure-A Report from the 2018 National Heart, Lung, and Blood Institute Workshop. Circulation. 2019;140:1205-1216.

28. Ronald JA, Kim BS, Gowrishankar G, et al. A PET Imaging Strategy to Visualize Activated T Cells in Acute Graft-versus-Host Disease Elicited by Allogenic Hematopoietic Cell Transplant. Cancer Res. 2017;77:2893-2902.

29. Franc BL, Goth S, MacKenzie J, et al. In Vivo PET Imaging of the Activated Immune Environment in a Small Animal Model of Inflammatory Arthritis. Mol Imaging. 2017;16:1536012117712638.

30. Levi J, Lam T, Goth SR, et al. Imaging of Activated T Cells as an Early Predictor of Immune Response to Anti-PD-1 Therapy. Cancer Res. 2019;79:3455-3465.

31. Levi J, Goth S, Huynh L, Lam T, Huynh TL, Schulte B, Packiasamy JA. (18)F-FAraG PET for CD8 Profiling of Tumors and Assessment of Immunomodulation by Chemotherapy. J Nucl Med. 2020.

32. Casares N, Pequignot MO, Tesniere A, et al. Caspase-dependent immunogenicity of doxorubicin-induced tumor cell death. J Exp Med. 2005;202:1691-1701.

33. Lake RA, Robinson BW. Immunotherapy and chemotherapy--a practical partnership. Nat Rev Cancer. 2005;5:397-405.

34. Galluzzi L, Buque A, Kepp O, Zitvogel L, Kroemer G. Immunological Effects of Conventional Chemotherapy and Targeted Anticancer Agents. Cancer Cell. 2015;28:690-714.

35. Wei SC, Meijers WC, Axelrod ML, et al. A genetic mouse model recapitulates immune checkpoint inhibitor-associated myocarditis and supports a mechanism-based therapeutic intervention. Cancer Discov. 2020.

36. Ashley N, Poulton J. Mitochondrial DNA is a direct target of anti-cancer anthracycline drugs. Biochem Biophys Res Commun. 2009;378:450-455.

37. Jung K, Reszka R. Mitochondria as subcellular targets for clinically useful anthracyclines. Adv Drug Deliv Rev. 2001;49:87-105.

38. Sivapackiam J, Kabra S, Speidel S, Sharma M, Laforest R, Salter A, Rettig MP, Sharma V. 68Ga-Galmydar: A PET imaging tracer for noninvasive detection of Doxorubicin-induced cardiotoxicity. PLoS One. 2019;14:e0215579.

39. McCluskey SP, Haslop A, Coello C, et al. Imaging of Chemotherapy-Induced Acute Cardiotoxicity with (18)F-Labeled Lipophilic Cations. J Nucl Med. 2019;60:1750-1756.

40. Bulten BF, Sollini M, Boni R, Massri K, de Geus-Oei LF, van Laarhoven HWM, Slart R, Erba PA. Cardiac molecular pathways influenced by doxorubicin treatment in mice. Sci Rep. 2019;9:2514.

41. Phillips NR, Sprouse ML, Roby RK. Simultaneous quantification of mitochondrial DNA copy number and deletion ratio: a multiplex real-time PCR assay. Sci Rep. 2014;4:3887.

42. Thut DP, Ahmed R, Kane M, Djekidel M. Variability in myocardial metabolism on serial tumor (18)F-FDG PET/CT scans. Am J Nucl Med Mol Imaging. 2014,4:346-353.

43. Maurer AH, Burshteyn M, Adler LP, Steiner RM. How to differentiate benign versus malignant cardiac and paracardiac 18F FDG uptake at oncologic PET/CT. Radiographics. 2011;31:1287-1305.

44. Radu CG, Shu CJ, Nair-Gill E, Shelly SM, Barrio JR, Satyamurthy N, Phelps ME, Witte ON. Molecular imaging of lymphoid organs and immune activation by positron emission tomography with a new [18F]-labeled 2′-deoxycytidine analog. Nat Med. 2008;14:783-788.

45. Manfredi JP, Holmes EW. Purine salvage pathways in myocardium. Annu Rev Physiol. 1985;47:691-705.

46. Xu M, Zhao M, Yang R, Zhang Z, Li Y, Wang J. Effect of dietary nucleotides on immune function in Balb/C mice. Int Immunopharmacol. 2013;17:50-56.

47. Dudzinska W, Lubkowska A, Dolegowska B, Safranow K, Jakubowska K. Adenine, guanine and pyridine nucleotides in blood during physical exercise and restitution in healthy subjects. Eur J Appl Physiol. 2010;110:1155-1162.

48. Gillies RJ, Kinahan PE, Hricak H. Radiomics: Images Are More than Pictures, They Are Data. Radiology. 2016;278:563-577.

49. Gillies RJ, Schabath MB. Radiomics Improves Cancer Screening and Early Detection. Cancer Epidemiol Biomarkers Prev. 2020;29:2556-2567.

50. Sun R, Limkin EJ, Vakalopoulou M, et al. A radiomics approach to assess tumour-infiltrating CD8 cells and response to anti-PD-1 or anti-PD-L1 immunotherapy: an imaging biomarker, retrospective multicohort study. Lancet Oncol. 2018;19:1180-1191.

51. Tang C, Hobbs B, Amer A, et al. Development of an Immune-Pathology Informed Radiomics Model for Non-Small Cell Lung Cancer. Sci Rep. 2018;8:1922.

52. van Griethuysen JJM, Fedorov A, Parmar C, et al. Computational Radiomics System to Decode the Radiographic Phenotype. Cancer Res. 2017;77:e104-e107.

53. pyradiomics.readthedocs.io/en/latest/features.html

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt% to about 5 wt%, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

1-9. (canceled)

10. A method of imaging cells in a heart of a subject responding to administration of a therapeutic agent comprising:
(a) administering the therapeutic agent;
(b) administering to the subject a compound having a formula: 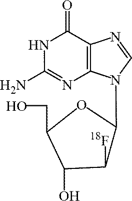
wherein a route of administration is selected so as to allow the compound to be phosphorylated by deoxycytosine kinase and deoxyguanosine kinase present in heart cells in the subject; and
(c) imaging the subject, wherein detecting the presence of the compound corresponds to the presence of cells; and
(d) correlating the observed presence of the compound in cells of the subject with the subject’s response to the therapeutic agent, wherein:
the subject is selected to be a patient diagnosed with cardiovascular disease;
the subject is selected to be a patient diagnosed with cancer;
the subject is selected to be a patient undergoing treatment for cardiovascular disease or cancer.

11. (canceled)

12. The method of claim 11, wherein correlating includes observations of presence of the compound in cancer cells and/or in lymph nodes.

13. The method of claim 11, wherein the therapeutic agent comprises at least one immune checkpoint inhibitor selected to affect CTLA-4 or a PD-1/PD-L1 blockade.

14. The method of claim 12, wherein the cancer cells are colon cancer cells.

15. The method of claim 11, wherein the method further comprises observing one or more images on a first date; observing one or more images on a second date; and comparing the images obtained on the first date with the images obtained on the second date so as to observe the subject’s response to administration of the therapeutic agent.

16. The method of claim 15, wherein an amount of time from the first date to the second date comprises at least one week or at least one month.

17. A method of imaging mitochondrial activity in cells in a subject comprising:
(a) administering to the subject a compound having a formula: 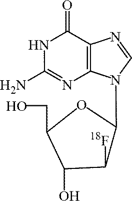
wherein a route of administration is selected so as to allow the compound to be phosphorylated by deoxycytosine kinase and deoxyguanosine kinase present in cells in the subject; and
(b) imaging the subject, wherein detecting the presence of the compound corresponds to the presence of mitochondrial activity; and
(c) imaging mitochondrial activity.

18. The method of claim 17, wherein the subject is one selected as suffering from a mitochondrial deficient disease.

19. The method of claim 17, wherein method is a method of screening the subject for mitochondrial dysfunction; and the mitochondrial dysfunction is a cardiovascular disease, a neuropsychiatric disease, or a neurodegenerative disease.

20. The method of claim 19, wherein the mitochondrial dysfunction is selected from the group consisting of myocardial perfusion, bipolar disorder, depression, schizophrenia Alzheimer’s disease, Parkinson’s disease, Friedreich’s ataxia, amyotrophic lateral sclerosis, Huntington’s disease, premature ageing, cardiomyopathy, a respiratory chain disorder, mtDNA depletion syndrome, myoclonus epilepsy, ragged-red fibers syndrome, myopathy encephalopathy lactic acidosis, stroke-like episodes, and optic atrophy.
